(19) United States
(12) Patent Application Publication
(10) Pub. No.: US 2003/0032582 A1
(43) Pub. Date: Feb. 13, 2003

(54) SPONTANEOUS MEMBRANE INSERTION

(76) Inventors: JENNIFER L. WAHLSTEN, ST. PAUL, MN (US); SUNDARAM RAMAKRISHNAN, NORTH ST. PAUL, MN (US); PATRICK M. SCHLIEVERT, EDINA, MN (US)

Correspondence Address: MARK S ELLINGER, FISH & RICHARDSON PC, 60 SOUTH SIXTH STREET, SUITE 3300, MINNEAPOLIS, MN, 55402

(21) Appl. No.: 09/465,714

(22) Filed: Dec. 17, 1999

Related U.S. Application Data
(62) Division of application No. 09/001,593, filed on Dec. 31, 1997, now abandoned.

(51) Int. Cl. A01N 37/18; A61K 38/00; C12P 21/06; C12P 21/04; A61K 39/00; A61K 39/38; A61K 39/085; A61K 39/09; C07K 5/00; C07K 7/00; C07K 16/00; C07K 17/00

(52) U.S. Cl. 514/2; 514/12; 530/324; 435/69.1; 435/69.7; 424/237.1

(57) ABSTRACT

The invention provides nucleic acid molecules encoding membrane-infiltrating polypeptides, host cells that express nucleic acid molecules that encode membrane-infiltrating polypeptides, and membrane-infiltrating polypeptides. Membrane-infiltrating polypeptides have the ability to insert spontaneously into cell membranes, and generally include a membrane-infiltrating amino acid sequence as well as another, selected, amino acid sequence heterologous to the membrane-infiltrating amino acid sequence. The invention also provides methods of treating mammals with cells that contain membrane-infiltrating polypeptides. Further, the invention provides truncated superantigen polypeptide encoding nucleic acid molecules as well as truncated superantigen polypeptides. These truncated superantigens can elicit an anti-tumor immune response without binding MHC II molecules, thus having a limited amount of toxicity. A separate group of truncated superantigens can bind MHC II molecules and inhibit the toxic effects associated with non-truncated superantigens.

### BACKGROUND

1. Technical Field

The invention relates to polypeptides having the ability to insert spontaneously into a cell membrane, nucleic acid molecules encoding these polypeptides, and the use of these polypeptides to treat disease.

2. Background Information

Cell surfaces contain a complex collection of membrane proteins. Most membrane proteins attach to the surface of cells through glycoinositol phospholipid (GPI)-anchors or membrane spanning domains. For GPI-anchored membrane proteins, the anchoring unit is a linear glycan having a phosphoethanolamine, three mannose residues, and a nonacetylated glucosamine linked to an inositol phospholipid. This unit is preformed in the endoplasmic reticulum (ER) and added en bloc to the protein during protein translocation across the ER membrane. For membrane proteins attached by membrane spanning domains, the attaching unit is usually a single or multiple hydrophobic amino acid sequences contained within the protein itself. Whether a GPI-anchor or a transmembrane domain, specific cellular machinery operating within the cell facilitates the incorporation of the membrane protein's attaching unit into a lipid membrane.

Gene therapy and genetic engineering allows scientists to manipulate and change the collection of membrane proteins found on a cell's surface. One common approach involves transfecting cells with DNA that encodes a membrane protein not normally found in those particular cells. Another approach involves adding a membrane attaching unit to an otherwise soluble protein. For example, the DNA encoding either a known sequence necessary for linking a GPI-anchor to a polypeptide or a hydrophobic transmembrane domain can be cloned into a DNA sequence encoding a soluble protein. The DNA encoding this newly designed membrane protein is then transfected into cells of interest for expression. Regardless of the specific approach, machinery within the transfected cell facilitates the insertion of the membrane attaching unit into a lipid membrane typically resulting in the appearance of the exogenous or newly designed membrane protein on the cell surface. Indeed, experiments fusing the extracellular domain of lymphocyte CD8 to the carboxy-terminal of the compliment decay accelerating factor, a known GPI-anchored protein, resulted in the surface expression of the fusion protein in a GPI-anchored fashion (Tykocinski ML et al., Proc. Natl. Acad. Sci. USA 85:3555-3559 (1988)). This technology depends on both the ability to transfect cells of interest as well as the ability of transfected cells to target the cloned protein to the cell surface. Not all cells have these abilities. For example, primary cultures, bone marrow progenitor cells, immune system cells, and tumors cells can be difficult to maintain and manipulate genetically in vitro.

Altering the protein composition of tumor cell surfaces is proving important for the treatment of cancer. Briefly, tumor cells often escape protective immune responses because they lack or down-regulate stimulatory surface antigens (Melief CJM, Adv. Cancer Res. 58:143-175 (1992)). For example, experimental results show that the introduction of immunostimulatory membrane proteins, such as B7-1, major histocompatibility class I molecules (MHC I), or MHC II molecules, to the surface of tumor cells induces anti-tumor immunity in mice (Baskar S, Cancer Immunol. Immunother. 43:165-173 (1996)). Other molecules, specifically superantigens, also elicit significant anti-tumor activity when present on the surface of tumor cells. Normally, they bind MHC II molecules outside the antigenic groove and stimulate T cells upon engaging particular T-cell receptor (TCR) Vβ elements (Marrack P and Kappler J, Science 248:705 (1990)). The formation of this ternary complex results in the activation of large numbers of T cells and monocytes with subsequent massive systemic cytokine release, most notably tumor necrosis factor (TNF) and interleukin-1 (IL-1; Parsonnet J et al., J. Infect. Dis. 151:514 (1985) and Jupin C et al., J. Exp. Med. 167:752 (1988)). Superantigens also stimulate cell-mediated cytotoxicity preferentially, but not exclusively, against MHC II-positive targets (Herrmann T et al., J. Immunol. 146:2504 (1991) and Lando PA et al., Cancer Immunol. Immunother. 33:231 (1991)). Interestingly, attaching superantigens, which are typically soluble proteins, to the surface of tumor cells that lack MHC II molecules induces a potent anti-tumor response with reduced toxicity upon administration to a host. Unfortunately, these approaches involve the transfection of cells with genes engineered such that the normally soluble protein is a membrane protein. Thus, practical application for treating human tumors is limited since transfecting cells is time consuming and primary tumor cells often do not grow well in vitro.

Two general methods that circumvent cell transfection have been devised to alter the collection of membrane proteins found on a cell's surface. The first method involves covalently attaching purified proteins to the cell surface by chemical treatment. Indeed, chemically linking the toxic shock syndrome toxin-I (TSST1), a pyrogenic toxin and superantigen produced by certain strains of Staphylococcus aureus and implicated as a causative factor in toxic shock syndrome (TSS; Bergdoll MS et al., Lancet 1:1017 (1981) and Schlievert PM et al., J. Infect. Dis. 143:509 (1981)), to murine tumor cells induced an anti-tumor immune response in mice. The attaching proteins and cells, however, are exposed to chemical conditions that may alter protein structure as well as cell viability. Further, chemically linking proteins to a surface is nonspecific resulting in countless different points of attachment and protein orientations. Some of these points of attachment or orientations may be unfavorable, rendering the protein functionless.

A second method of altering the cell surface membrane protein composition originates from the discovery that purified GPI-anchored membrane proteins containing intact GPI-anchors can reinsert themselves into cell membranes spontaneously after simple incubation with living cells. Thus, any protein can be designed to contain a consensus sequence necessary for linking a GPI-anchor to a polypeptide. This method, however, requires that the proteins be expressed in an appropriate eukaryotic cell since prokaryotic cells do not modify proteins with necessary carbohydrate moieties. In addition, the protein must be purified such that the GPI-anchor remains intact prior to adding it to the desired cell for membrane insertion.

### SUMMARY

The invention is based on the discovery of polypeptides that spontaneously insert into cell membranes. Specifically, the invention relates to polypeptides containing a membrane-infiltrating sequence that allows the polypeptide to insert spontaneously into cell membranes. These membrane-infiltrating polypeptides provide a means for changing the composition of a cell surface without the need of transfecting the cell with nucleic acid. In addition, the amount of membrane-infiltrating polypeptide present on the cells can be accurately measured, controlled, and adjusted. Unlike GPI-anchoring strategies, these membrane-infiltrating polypeptides do not require post-translational modifications, such as the addition of carbohydrate moieties, as a means of cell surface attachment. Thus, these membrane-infiltrating polypeptides can be expressed in bacteria, avoiding the difficulties associated with protein expression and purification using eukaryotic cells. The invention also provides membrane-infiltrating polypeptides containing amino acid sequences that facilitate purification of membrane-infiltrating polypeptides. Thus, large amounts of relatively pure polypeptide can be produced quickly. The invention also relates to nucleic acid molecules encoding membrane-infiltrating polypeptides, host cells expressing membrane-infiltrating polypeptides, and cells containing membrane-infiltrating polypeptides. Cells containing membrane-infiltrating are particularly useful and unique in that they do not require nucleic acid encoding membrane-infiltrating polypeptides. Thus, using membrane-infiltrating polypeptides to alter the cell surface composition of cells avoids the need for nucleic acid transfection methods. Further, cells altered using membrane-infiltrating polypeptides are not required to contain exogenous nucleic acid sequences, such as viral expression vectors, to promote the expression of the desired polypeptide. This invention also relates to the treatment of a patient with cells containing membrane-infiltrating polypeptides. Specifically, tumor cells containing a membrane-infiltrating superantigen polypeptide induces anti-tumor immune responses after administration to a host.

In general, the invention features a nucleic acid molecule encoding a polypeptide that includes a membrane-infiltrating amino acid sequence and a selected amino acid sequence, with the selected amino acid sequence being heterologous to the membrane-infiltrating amino acid sequence. The polypeptide can further include an amino acid sequence that facilitates purification of the polypeptide. The membrane-infiltrating amino acid sequence typically constitutes a transmembrane domain. Useful transmembrane domains include, for example, the transmembrane domain of c-erb-B-2 (e.g., SEQ ID NO:5). The selected amino acid sequence can be a superantigen, for example a bacterial toxin such as toxic shock syndrome toxin. Such sequences can include whole superantigen molecules (e.g., the entire toxic shock syndrome toxin, SEQ ID NO:3) as well as fragments having defined activities (e.g. fragments having reduced MHC II binding (e.g., SEQ ID NO:4, which is a toxic shock syndrome toxin fragment lacking MHC II binding capability).

In another aspect, the invention features a host cell including a nucleic acid molecule encoding a polypeptide. The polypeptide includes a membrane-infiltrating amino acid sequence and a selected amino acid sequence heterologous to the membrane-infiltrating amino acid sequence. The polypeptide is expressed by the host cell, which can be either a prokaryotic cell or a eukaryotic cell.

In another aspect, the invention features a polypeptide that includes a membrane-infiltrating amino acid sequence and a selected amino acid sequence heterologous to the membrane-infiltrating amino acid sequence.

In still another aspect, the invention features a cell that includes a polypeptide, the polypeptide including a membrane-infiltrating amino acid sequence and a selected amino acid sequence heterologous to the membrane-infiltrating amino acid sequence. Such cells need not be expressing the corresponding gene, i.e., the cells can be free of nucleic acid encoding the polypeptide. The cells can be eukaryotic, e.g., mammalian, cells, and can be tumor cells in some circumstances.

In a further aspect, the invention features a method of altering the surface of a cell, involving contacting the cell with a polypeptide. The polypeptide includes a membrane-infiltrating amino acid sequence and a selected amino acid sequence. In this aspect of the invention, the selected amino acid sequence can, but need not, be heterologous to the membrane-infiltrating amino acid sequence.

In another aspect, the invention features a method of treating a mammal by administering cells to the mammal. These cells include a polypeptide that itself includes a membrane-infiltrating amino acid sequence and a selected amino acid sequence heterologous to the membrane-infiltrating amino acid sequence. The cells can be free of nucleic acid encoding the polypeptide. The cells can be tumor cells, and can be obtained from the same mammal if desired. The polypeptide can include a superantigen.

In another aspect, the invention features a nucleic acid molecule encoding a truncated superantigen lacking the ability to bind MHC II molecules while retaining the ability to induce an anti-tumor activity. The truncated superantigen can include the amino acid sequence of SEQ ID NO:4. Also included are nucleic acid molecules that encode truncated superantigens that, in contrast, have the ability to bind MHC II molecules but that inhibit the toxic effects of non-truncated superantigens. An example of such a truncated superantigen is a superantigen that includes the amino acid sequence of SEQ ID NO: 11. The invention also includes the polypeptides encoded by these nucleic acid molecules (e.g., SEQ ID NOs:4 and 11).

In a further aspect, the invention features a method of treating a mammal involving administering to the mammal a truncated superantigen that binds MHC II molecules and inhibits the toxic effects of a non-truncated superantigen.

In a still further aspect, the invention features a method of treating a mammal involving administering to the mammal a truncated superantigen that lacks the ability to bind MHC II molecules and inhibits the toxic effects of a non-truncated superantigen.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic depicting the HTSST1-TM and HT84-TM polypeptides. Arrows indicate relative positions of the oliogonucleotide primers used to create the HTSST1-TM and HT84-TM sequences by splice overlap polymerase chain reaction (PCR). Boxes are not proportional.

FIG. 2 depicts the amino acid sequence of TSST1-TM (SEQ ID NO: 1).

FIG. 3 depicts the amino acid sequence of T84-TM (SEQ ID NO:2).

FIG. 4 is a hydropathy chart depicting the hydrophilicity of TSST1-TM.

FIG. 5 is a hydropathy chart depicting the hydrophilicity of T84-TM.

FIG. 6 is a representative Coomassie Brilliant Blue-stained sodium dodecylsulfate-(SDS) polyacrylamide gel depicting the expression of HTSST1, HTSST1-TM, and HT84-TM in host BL21 (DE3) pLysS cells with and without IPTG induction. Lanes 1 and 2 are samples from uninduced and induced, HTSST1-containing bacteria, respectively. Lanes 3 and 4 are samples from uninduced and induced, HTSST1-TM-containing bacteria, respectively. Lanes 5 and 6 are samples from uninduced and induced, HT84-TM-containing bacteria, respectively.

FIG. 7 is a representative Coomassie Brilliant Blue-stained SDS-polyacrylamide gel depicting purified HTSST1-TM (5 μg) and HT84-TM (2 μg).

FIG. 8 is a chart depicting the flow cytometry results of MHC II expression by Raji and MA 148 cells. Positive detection of MHC II expression on Raji was reflected by an increase in fluorescence intensity or shifting of the test black peak verses the control gray peak. Result generated using MA 148 cells showed no shifting of the black peak verses control.

FIG. 9 is a chart depicting the flow cytometry results that demonstrate the spontaneous membrane insertion of HTSST1-TM into cell membranes of MA 148 cells. Controls were HTSST1-treated cells as well as cells stained with secondary antibody only.

FIG. 10 is a chart depicting the flow cytometry results that demonstrate the spontaneous membrane insertion of HTSST1-TM into cell membranes of MHC II-negative tumor cell lines, MA 148 (7500 events), RJ2.2.5 (104 events), LLC (7500 events), and EL4 (2000 events). Fluorescence was measured on cells exposed to serum-free media ( . . . ), 0.75 μM HTSST1 ( ), or HTSST1-TM (solid). HTSST1 binding to EL4 cells was not tested.

FIG. 11 is a representative set of indirect immunofluorescence micrographs depicting HTSST1-TM on the surface of MA 148 cells. Panels A and B depict MA 148 cells incubated in media containing 2.5% serum proteins without HTSST1-TM. Panels C and D depict MA 148 cells incubated in media containing 2.5% serum proteins with 1.0 μM HTSST1-TM for 4 hours. Panels A and C are phase-contrast exposures, whereas panels B and D are fluorescence exposures. The bar represents a length of 30 μm.

FIG. 12 is a representative set of indirect immunofluorescence micrographs depicting the effects of serum proteins on the insertion of HTSST1-TM into MA 148 cell membranes. Top panels are fluorescence exposures, whereas bottom panels are phase-contrast exposures.

FIG. 13 is a bar graph depicting the stimulation of PBL proliferation in vitro after incubation with MA 148 cells coated with either HTSST 1-TM or HT84-TM. The asterisk indicates a significance of p<0.004 versus uncoated MA 148 cells.

FIG. 14 is a bar graph depicting the anti-tumor response results for mice immunized with HTSST1-TM- or HT84-TM-coated P815 cells. Values on the y-axis represent the average bisecting tumor diameters for each treatment group. The asterisk indicates a significance of p≦0.03 versus P815 immunized control.

FIG. 15 is a schematic depicting various TSST1 constructs. The presence of an amino terminal histidine tag (H-), a Kemptide sequence (K-), or a cysteine residue (cys) is indicated. Boxes are not proportional.

FIG. 16 is a graph plotting the bound radioactivity for the indicated TSST1 construct verses concentration.

FIG. 17 is a graph plotting incorporated radioactivity for cells treated with the indicated TSST1 construct verses concentration.

FIG. 18 is a graph plotting the specific cell lysis verses the effector:target cell ratio.

FIG. 19 is a graph plotting the average bisecting tumor diameter verses time elapsed after inoculation for the indicated treatments.

### DETAILED-DESCRIPTION

The invention provides nucleic acid molecules encoding membrane-infiltrating polypeptides, host cells that express nucleic acid molecules that encode membrane-infiltrating polypeptides, and membrane-infiltrating polypeptides. In addition, the invention provides cells that contain membrane-infiltrating polypeptides as well as methods of making such cells. The invention also provides methods of treating mammals with cells that contain membrane-infiltrating polypeptides. Further, the invention provides truncated superantigen polypeptide encoding nucleic acid molecules as well as truncated superantigen polypeptides. These truncated superantigens are useful and unique since they can elicit an anti-tumor immune response without binding MHC II molecules, thus having a limited amount of toxicity. A separate group of truncated superantigens are useful and unique in that they bind MHC II molecules and inhibit the toxic effects associated with non-truncated superantigens. Nucleic Acid Molecules Encoding Membrane-infiltrating Polypeptides

Nucleic acid molecules encoding membrane-infiltrating polypeptides can be cDNA, genomic DNA, synthetic DNA, or RNA, and can be double-stranded or single-stranded. Fragments of these molecules are also considered within the scope of the invention, and can be produced, for example, by polymerase chain reaction (PCR) or generated by treatment with one or more restriction endonucleases. Ribonucleic acid (RNA) molecules can be produced by in vitro transcription. In addition, the nucleic acid molecules of the invention can be any length provided that the molecule encodes a membrane-infiltrating polypeptide.

Nucleic acid molecules encoding membrane-infiltrating polypeptides contain at least two heterologous nucleic acid sequence components. One nucleic acid sequence component encodes a membrane-infiltrating amino acid sequence and the other encodes a selected amino acid sequence. A membrane-infiltrating amino acid sequence is any amino acid sequence having the ability to insert itself spontaneously into a cell membrane. For example, a membrane-infiltrating amino acid sequence can be a transmembrane domain (TMD) including, without limitation, the TMD of c-erb-B-2. Typically, TMDs are long enough to span a membrane, hydrophobic, and α-helical in structure. Membrane-infiltrating amino acid sequences can be sequences found in nature as well as sequences that are engineered. Thus, any improvements, substitutions, truncations, and alterations to a naturally occurring TMD or any other sequence having membrane-infiltrating capability is within the scope of the invention. Spontaneous membrane insertion means that the process of membrane insertion is enabled by simple interaction between the membrane-infiltrating amino acid sequence of a membrane-infiltrating polypeptide, and the membrane, upon incubation. For example, membrane-infiltrating polypeptides of the present invention will spontaneously insert into the membranes of living cells when applied externally in an appropriate medium.

A selected amino acid sequence can be any length and have any amino acid sequence. Thus, a selected amino acid sequence of the invention includes, without limitation, amino acid sequences found in sequence databases such as Genebank® as well as one or more full-length polypeptides, mutant polypeptides, recombinant polypeptides, natural polypeptides, synthetic polypeptides, polypeptide fragments, and polypeptides that are preproteins or proproteins.

The arrangement of the two nucleic acid sequence components within the nucleic acid molecule of the invention can be any arrangement providing the membrane-infiltrating amino acid sequence of the expressed membrane-infiltrating polypeptide remains capable to insert into a cell membrane. Typically, the membrane-infiltrating nucleic acid sequence component is located terminally, for example, at the 3′ or 5′ terminal end of the coding region. In addition, the membrane-infiltrating amino acid sequence can contain a single TMD that spans the membrane one time, or multiple TMDs that span the membrane several times. Further, the membrane-infiltrating amino acid sequence can be hydrophobic and alpha-helical in structure.

The cell membranes in which membrane-infiltrating polypeptides insert themselves can include any membrane barrier of a cell including, without limitation, plasma membranes, vesicular membranes, and organellar membranes, as well as artificial membranes such as those commonly produced from natural or synthetic lipid extracts. Thus, cell membranes from both prokaryotic and eukaryotic cells are considered within the scope of the invention.

The nucleic acid molecules of the invention also encompass: (a) expression vectors that contain any of the foregoing membrane-infiltrating polypeptide encoding sequences; (b) expression vectors that contain any of the foregoing membrane-infiltrating polypeptide encoding sequences operatively associated with a regulatory element (examples of which are given below) that directs the expression of the coding sequences; and (c) expression vectors containing, in addition to sequences encoding membrane-infiltrating polypeptide, nucleic acid sequences that are unrelated to nucleic acid sequences that encode membrane-infiltrating polypeptides, such as molecules encoding reporters or markers, such as those described below.

Regulatory elements include, without limitation, inducible and non-inducible promoters, enhancers, operators, and other elements that are known to those skilled in the art, and which drive or otherwise regulate gene expression. Such regulatory elements include, without limitation, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast a-mating factors.

Similarly, the nucleic acid molecules of the invention can form part of a hybrid molecule encoding additional polypeptide sequences, for example, sequences that function as a reporter or marker. Examples of reporter and marker genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). As with many of the standard procedures associated with the practice of the invention, skilled artisans will be aware of additional useful reagents, for example, additional sequences that can serve the function of a reporter or marker. Generally, the hybrid polypeptides will include a first portion and a second portion: the first portion being a membrane-infiltrating polypeptide and the second portion being, for example, a reporter described above or other molecules such as an immunoglobulin constant region.

Host Cells Expressing Membrane-Infiltrating Polypeptides

Any cell that contains and expresses a membrane-infiltrating polypeptide encoding nucleic acid molecule is considered a host cell within the scope of the invention. This includes both eukaryotic and prokaryotic cells. In addition, these cells can be, without limitation, transiently, stably, or virally transfected. Thus, the means of introducing the membrane-infiltrating polypeptide encoding nucleic acid molecules into a host cell is irrelevant providing the host cell expresses a membrane-infiltrating polypeptide. For example, heat shock, electroporation, calcium-phosphate precipitation, lipofection, or viral infection can be used to introduce membrane-infiltrating polypeptide encoding nucleic acid molecules into host cells for expression. Further, the nucleic acid molecules used to transfect the host cells can be naked or part of an expression vector as described above.

The expression systems that may be used for purposes of the invention include, but are not limited to microorganisms such as bacteria (for example, E. Coli and B. Subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention; insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing membrane-infiltrating polypeptide encoding nucleic acid sequences; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected depending upon the use intended for the membrane-infiltrating polypeptide being expressed. For example, when a large quantity of a membrane-infiltrating polypeptide is to be produced, vectors that are capable of directing the expression of high levels of fusion polypeptide products that are readily purified may be desirable. Such vectors include, but are not limited to, the E. coli expression vector pUR278 (Ruther et al., EMBO J., 2:1791 (1983)), in which the coding sequence of the insert may be ligated individually into the vector in frame with the lacZ coding region so that a fusion polypeptide is produced; pIN vectors (Inouye and Inouye, Nucleic Acids Res., 13:3101-3109 (1985) and Van Heeke and Schuster, J. Biol. Chem., 264:5503-5509 (1989)); and the like. pGEX vectors may also be used to express foreign polypeptides as fusion polypeptides with glutathione S-transferase (GST). In general, such fusion polypeptides are soluble and can easily be purified from lysed cells by adsorption to glutathione-agarose beads followed by elution in the presence of free glutathione. The pGEX vectors are designed to include thrombin or factor Xa protease cleavage sites so that the cloned target polypeptide can be released from the GST moiety. The pET 17b Histag expression vector (pET 17bH) may also be used to express polypeptides. In this case, ten histidine residues are added to the amino-terminus of the polypeptide, providing an easy and convenient method of purification using Ni²⁺ adsorption (Mohanraj D et al., J. Immunol. Meth. 182:165-175 (1995)).

In an insect system, Autographa californica nuclear polyhidrosis virus (AcNPV) can be used as a vector to express foreign nucleic acid molecules. The virus grows in Spodoptera frugperda cells. The coding sequence of the insert may be cloned individually into non-essential regions (for example, the polyhedrin gene) of the virus and placed under control of an AcNPV promoter (for example, the polyhedrin promoter). Successful insertion of the coding sequence will result in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted nucleic acid sequence is expressed. (For example, see Smith et al., J. Virol., 46:584 (1983); Smith, U.S. Pat. No. 4, 215,051).

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric molecule may then be inserted in the adenovirus genome by in vitro or in vivo recombination. Insertion in a non-essential region of the viral genome (for example, region E1 or E3) will result in a recombinant virus that is viable and capable of expressing a membrane-infiltrating polypeptide in infected hosts (for example, see Logan and Shenk, Proc. Natl. Acad. Sci. USA, 81:3655-3659 (1984)). Specific initiation signals may also be required for efficient translation of inserted nucleic acid molecules. These signals include the ATG initiation codon and adjacent sequences.

In cases where an entire gene or cDNA, including its own initiation codon and adjacent sequences, is inserted into the appropriate expression vector, no additional translational control signals may be needed. In cases where only a portion of the coding sequence is inserted, however, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol., 153:516-544 (1987)).

In addition, a host cell strain may be chosen that modulates the expression of the inserted sequences, or modifies and processes the membrane-infiltrating polypeptide in the specific fashion desired. Such modifications (for example, glycosylation) and processing (for example, cleavage) of polypeptide products may be important for the function of the polypeptide. Different host cells have characteristic and specific mechanisms for the post-translational processing and modification of polypeptides and gene products. Appropriate cell lines or host systems can be chosen to ensure the correct modification and processing of the foreign polypeptide expressed. To this end, eukaryotic host cells that possess the cellular machinery for proper processing of the primary transcript, glycosylation, and phosphorylation of the gene product may be used.

For long-term, high-yield production of membrane-infiltrating polypeptides, stable expression is preferred. For example, cell lines that stably express the membrane-infiltrating polypeptide encoding nucleic acid molecules described above may be engineered. Rather than using expression vectors that contain viral origins of replication, host cells can be transformed with DNA controlled by appropriate expression control elements (for example, promoter, enhancer sequences, transcription terminators, and polyadenylation sites), and a selectable marker. Following the introduction of the foreign DNA, engineered cells can be allowed to grow for 1-2 days in an enriched medium, and then switched to a selective medium. The selectable marker in the recombinant plasmid confers resistance to the selection agent. Cells that stably integrate the plasmid into their chromosomes can be selected and grown to form foci that in turn can be cloned and expanded into cell lines. This method can be used to engineer cell lines that express membrane-infiltrating polypeptides.

A number of selection systems can be used. For example, the herpes simplex virus thymidine kinase (Wigler et al., Cell, 11:223 (1977)), hypoxanthine-guanine phosphoribosyltransferase (Szybalska and Szybalski, Proc. Natl. Acad. Sci. USA, 48:2026 (1962)), and adenine phosphoribosyltransferase (Lowy, et al., Cell, 22:817 (1980)) genes can be employed in tk⁻, hgprt⁻, or aprt⁻ cells respectively. Also, anti-metabolite resistance can be used as the basis of selection for the following genes: dhfr, which confers resistance to methotrexate (Wigler et al., Proc. Natl. Acad. Sci. USA 77:3567 (1980) and O'Hare et al., Proc. Natl. Acad. Sci. USA, 78:1527 (1981)); gpt, which confers resistance to the aminoglycoside G418 (Colberre-Garapin et al., J. Mol. Biol. 150:1 (1981)); and hygro, which confers resistance to hygromycin (Santerre et al., Gene, 30:147 (1984)).

Alternatively, any fusion polypeptide may be readily purified by utilizing an antibody specific for the fusion polypeptide being expressed. For example, a system described by Janknecht et al. allows for the ready purification of non-denatured fusion polypeptides expressed in human cell lines (Proc. Natl. Acad. Sci. USA, 88:8972-8976 (1991)). In this system, the nucleic acid molecule of interest is subcloned into a vaccinia recombination plasmid such that the molecule's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged polypeptides are selectively eluted with imidazole-containing buffers.

Membrane-Infiltrating Polypeptides

Membrane-infiltrating polypeptides contain at least two heterologous amino acid sequence components. One amino acid sequence component is a membrane-infiltrating amino acid sequence and the other is a selected amino acid sequence. As described above, a membrane-infiltrating amino acid sequence is any amino acid sequence having the ability to insert itself spontaneously into a cell membrane and a selected amino acid sequence is any amino acid sequence having any length. Thus, membrane-infiltrating polypeptides include those encoded by any of the membrane-infiltrating polypeptide encoding nucleic acid molecules described herein as well as fragments, mutants, and truncated forms. In addition, membrane-infiltrating polypeptides can be synthetic polypeptides as well as fusion polypeptides. For example, a membrane-infiltrating amino acid sequence can be covalently or non-covalently attached to a selected amino acid sequence that was separately translated. In this case, the membrane-infiltrating polypeptide was not transcribed or translated as a single unit.

The arrangement of the two amino acid sequence components within a membrane-infiltrating polypeptide can be any arrangement providing the membrane-infiltrating amino acid sequence of the expressed membrane-infiltrating polypeptide remains able to insert into a cell membrane. Typically, the membrane-infiltrating amino acid sequence component is located terminally, for example, at the amino or carboxy terminus of the polypeptide. As described above, the membrane-infiltrating amino acid sequence can contain a single TMD that spans the membrane one time or multiple TMDs that span the membrane several times. Further, the membrane-infiltrating amino acid sequence can be, without limitation, hydrophobic and alpha-helical in structure.

In addition to a membrane-infiltrating amino acid sequence and a selected amino acid sequence, the membrane-infiltrating polypeptides of the invention can contain other molecules including, without limitation, protein modifications, cofactors, metal ions, lipids, nucleic acids, fluorescent dyes, radioactive compounds, energy reactive compounds, and separate non-covalently attached polypeptide subunits.

Preferred membrane-infiltrating polypeptides of the invention are substantially pure polypeptides. For example, polypeptides that have an average relative purity greater than 85 percent as determined by densitometric measurement of Coomassie Brilliant Blue R-250-stained 12% SDS-polyacrylamide gels is considered substantially pure. Less pure membrane-infiltrating polypeptides, however, are also considered within the scope of the invention, especially since the presence of non-membrane-infiltrating polypeptides does not significantly interfere with the ability of membrane-infiltrating polypeptides to insert spontaneously into a cell membrane. Thus, crude protein extracts from host cells expressing a membrane-infiltrating polypeptide are within the scope of the invention.

As discussed above, membrane-infiltrating polypeptides can contain additional amino acid sequences that facilitate the purification of the membrane-infiltrating polypeptide. For example, membrane-infiltrating polypeptides can contain multiple sequentially located histidine residues (preferably six to ten histidine residues) or the GST amino acid sequence.

Cells Containing Membrane-infiltrating Polypeptides

Any cell can contain a membrane-infiltrating polypeptide. Cells within the scope of the invention include prokaryotic and eukaryotic cells that have been incubated with a solution containing membrane-infiltrating polypeptides such that the membrane-infiltrating polypeptides insert spontaneously into the cell's membrane. These coated cells can also contain nucleic acid molecules that encode a membrane-infiltrating polypeptide, although this is not required. Thus, cells can be both transfected with membrane-infiltrating polypeptide encoding nucleic acid and coated with membrane-infiltrating polypeptides. In addition, a cell can contain a single membrane-infiltrating polypeptide or several different membrane-infiltrating polypeptides.

Cells containing membrane-infiltrating polypeptides can also express other exogenous polypeptides. For example, a cell can be extracted from a mammal, cultured, transfected with DNA encoding an exogenous polypeptide, and then coated with a membrane-infiltrating polypeptide. Thus, a cell that contains a membrane-infiltrating polypeptide that was not synthesized within that particular cell is considered within the scope of the invention. In addition, cells containing membrane-infiltrating polypeptides can be irradiated or chemically treated such that they can no longer divide. This is particularly useful when using membrane-infiltrating polypeptide-containing cells to treat diseases. For example, primary tumor cells can be extracted from a cancer patient, coated with a membrane-infiltrating polypeptide that has anti-tumor activity, irradiated, and injected into the same patient. In this case, the cells containing membrane-infiltrating polypeptides will be unable to divide, but will be a useful vehicle for delivering the membrane-infiltrating polypeptides that triggers an anti-tumor response. Further, the cells need not be alive provided that they contain a membrane-infiltrating polypeptide. For example, cells can be coated with a membrane-infiltrating polypeptide and then chemically fixed, dehydrated, frozen, or immobilized such that the cell is no longer viable.

Triggering an Immunostimulatory Response

The invention provides a useful and unique method of treating mammals such that an immune response is stimulated. Specifically, cells containing a membrane-infiltrating polypeptide are administered to a mammal under conditions that trigger an immune response. The selected amino acid sequence component of the membrane-infiltrating polypeptide can be a specific target antigen including, without limitation, viral antigens, bacterial antigens, tumor antigens, and parasitic antigens. The immune response triggered can be specifically directed toward the particular antigen, for example, the production of anti-antigen antibodies. The selected amino acid sequence component of the membrane-infiltrating polypeptide also can be a polypeptide sequence known to have immunostimulatory activity including, without limitation, superantigens, cytokines, and accessory molecules such as B7, CD4, and CD8. Membrane-infiltrating polypeptides having immunostimulatory activity typically will contain the immunostimulatory region of a superantigen as the selected amino acid component. For example, a membrane-infiltrating polypeptide can contain the entire coding region of a superantigen or a truncated or mutated version that retains the ability to stimulate an immune response. Using membrane-infiltrating polypeptides to trigger an immune response is particularly beneficial since: (a) these polypeptides circumvent the need to transfect cells with DNA; (b) the amount of membrane-infiltrating polypeptide administered can be accurately measured and controlled; and (c) the length of time that the administered cells contain the membrane-infiltrating polypeptide is limited.

Superantigens are primarily polypeptides of viral or bacterial origin and are capable of simultaneous binding to MHC II molecules and to the TCR VP chain. The binding leads to activation of T cells and lysis of the MHC II bearing cells. The moderate degree of polymorphism of the binding part of the VP chain causes a relatively large portion of T cells to be activated when contacted with a superantigen, compared to activation through normal antigen-processing.

Initially, the superantigen concept was associated with various staphylococcal enterotoxins (SEA, SEB, SEC₁, SEC₂, SED, and SEE). Later, additional superantigens were discovered. Examples include staphylococcal enterotoxin H (SEH), TSST1, exfoliating toxins (Exft) that are associated with scaled skin syndrome, Streptococcal pyrogenic exotoxin A, B and C (SPEA, SPEB, and SPEC), mouse mammary tumor virus proteins, Streptococcal M proteins, and Clostridial perfringens enterotoxin (CPET) among others. For review of superantigens and their properties, see Kotzin et al., Adv. Immunol. 54:99-166 (1993).

Superantigen-based therapeutic approaches have been suggested for the treatment of various diseases, with curative effects being accomplished through a general activation of the immune system (Kalland et al., WO 9104053; Terman et a., WO 9110680; Terman et al., WO 9324136; Newell et al., Proc. Natl. Acad. Sci. USA 88:1074-1078 (1991)). Now, these approaches can be combined with the discoveries described herein to attach superantigen polypeptides as well as other immunoregulatory polypeptides such as cytokines onto the surfaces of cells, enabling delivery of exogenous polypeptides in the context of cells without the use of nucleic acid transfection methods.

A specific immune response also can be generated using cells containing two general elements: an immunostimulatory membrane-infiltrating polypeptide and a specific target antigen. The immunostimulatory membrane-infiltrating polypeptide, the first element, has the ability to stimulate and induce an immune response, whereas the second element provides the specific antigen to be targeted by the induced immune response. This specificity can be determined by the antigens present on the cells administered to the mammal. Specifically, an antigen can be coated onto the administered cell as a separate membrane-infiltrating polypeptide, expressed by the administered cell endogenously, or expressed by the administered cell from a transfected exogenous nucleic acid sequence. In the former two cases, the tedious and sometimes difficult process of transfecting cells is circumvented. For example, cells that are normally not recognized by an individual's immune system can be collected, coated with an immunostimulatory membrane-infiltrating polypeptide, and administered back to the individual such that an immune response is triggered having specificity for the specific cells that were coated. In this case, the specific antigen or antigens were provided endogenously by the administered cell. Further, the immunostimulatory membrane-infiltrating polypeptide can contain any polypeptide sequence known to have immunostimulatory activity including superantigens as described above.

Using this method to treat cancer patients is particularly beneficial since tumor cells can be difficult to culture and transfect. For example, tumor cells from a cancer patient can be collected, coated with an immunostimulatory membrane-infiltrating polypeptide, and injected back into the patient such that the immunostimulatory membrane-infiltrating polypeptide activates immune cells that target and destroy both the specific tumor cells containing the immunostimulatory membrane-infiltrating polypeptide and similar tumor cells that do not contain the immunostimulatory membrane-infiltrating polypeptide. As indicated, administering a cell that contains both an immunostimulatory membrane-infiltrating polypeptide and a specific target antigen can trigger an immune response against any specific antigen. Specific target antigens can be, for example, viral, bacterial, tumor, or parasitic antigens.

The cells administered can contain a single membrane-infiltrating polypeptide or many different membrane-infiltrating polypeptides. In addition, the administered cells can include different groups of cells each containing their own set of membrane-infiltrating polypeptides. For example, one group of cells, which express high levels of a particular cytokine, can be coated with a membrane-infiltrating polypeptide that contains a tumor antigen-specific antibody as its selected amino acid sequence component; whereas another group of cells, which are tumor cells, can be coated with an immunostimulatory membrane-infiltrating polypeptide. Further, these different groups of cells can be administered at the same or different times.

The cells administered to a mammal can be any cell including, without limitation, neurons, glial cells, tumor cells, skin cells, immune cells, bacterial cells, cell lines, and stem cells as well as mixtures of different types of cells, provided they contain a membrane-infiltrating polypeptide. In addition, the cells can be derived from the same individual mammal that is to be treated, a histocompatible mammal of the same species, or an entirely distinct species. For example, prokaryotic cells can be coated with a membrane-infiltrating polypeptide and administered to a human.

The amount of membrane-infiltrating polypeptide coated onto the cells to be administered can be any amount that stimulates an immune response without inducing significant toxicity. This amount can be determined by serially diluting the membrane-infiltrating polypeptide used to coat cells and measuring, after administration, the amount of toxicity as well as the ability of the administered cells to elicit an immune response. Likewise, the number of coated cells administered or the amount of target antigen also coated onto the cells can be any amount that stimulates an immune response without inducing significant toxicity. Again, these amounts can be determined by serially diluting either the number of cells administered or the amount of target antigen coated onto the cell membrane.

The mode of administration can be any route including, without limitation, intravenous, intradermal, subcutaneous, and intraperitoneal as well as oral, nasal, and the like, providing an immune response is triggered. The cells can be administered in a physiological saline solution or any other appropriate carrier for administering living, irradiated, or dead cells to a mammal which will be apparent to a skilled artisan. An immune response is any response that activates immune cells such that the degree of protective immunity provided by the immune system to the host is increased. This includes, without limitation, the production of antibodies, the proliferation and activation of cytolytic lymphocytes, and the mobilization of phagocytic cells.

Triggering an Immunosuppressive Response

The invention also provides a method of treating a mammal such that an immune response is suppressed. Specifically, cells containing a membrane-infiltrating polypeptide are administered to a mammal under conditions that trigger the suppression of an immune response. The selected amino acid sequence component of the membrane-infiltrating polypeptide can be a polypeptide sequence known to have immunosuppressive activity such as an immunosuppressive cytokine polypeptide sequence. Interestingly, membrane-infiltrating polypeptides containing the same immunostimulatory superantigens described above can also be used to suppress immune responses when administered to a mammal at specific concentrations. Thus, an immunosuppressive membrane-infiltrating polypeptide can contain the identical immunostimulatory region of a superantigen as the selected amino acid component because the conditions under which the superantigen-containing membrane-infiltrating polypeptide is administered to a mammal can determine whether an immune response is stimulated or suppressed.

The amount of superantigen-containing membrane-infiltrating polypeptide coated onto the cells to be used to trigger immunosuppression can be any amount that suppresses an immune response without inducing significant toxicity. This amount can be determined by serially diluting the concentration of membrane-infiltrating polypeptide used to coat cells and measuring, after administration, the amount of toxicity as well as the ability of the administered cells to trigger an immunosuppressive response. Immunosuppressive responses include, without limitation, T cell anergy, clonal deletion, and the increased release of immunosuppressive molecules such as nitric oxide and TNF-α . Likewise, the number of coated cells administered or the amount of target antigen also coated onto the cells can be any amount that triggers immunosuppression without inducing significant toxicity. Again, these amounts can be determined by serially diluting either the number of cells administered or the amount of target antigen coated onto the cell membrane.

Typically, low concentrations of superantigen stimulate immune responses, whereas high concentrations suppress them. For example, tumor cells such as P815 cells coated with 1.67 μM HTSST1-TM, a membrane-infiltrating polypeptide that contains the entire TSST1 amino acid sequence and ten amino-terminal histidine residues, have a diminished anti-tumor response when compared to the same cells coated with less HTSST1-TM, possibly because of T cell anergy or deletion. Since TSST1 can directly stimulate macrophages through MHC II engagement (Hauschildt S, Eur. J Immunol. 23:2988-2992 (1993); Fleming SD, Infect. Immun. 59:4049-4055 (1991); and Buyalos RP et al., Obstet. Gynecol. 78:182-186 (1991)), high TSST1-TM concentrations can cause the excessive stimulation of macrophages. Macrophage stimulation is known to have immunosuppressive consequences, at least partly mediated by nitric oxide and TNF-α, that can interfere with a productive anti-tumor response (Mills C et al., J Immunol. 149:2709-2714 (1992); Alleva DG, J. Immunol. 153:1674-1686 (1994); and Scott MT, Cell. Immunol. 5:469-479 (1972)).

Thus, creating TSST1 membrane-infiltrating polypeptides represents a novel method for passively introducing both immunostimulatory or immunosuppressive polypeptides onto cells.

Immunosuppressive responses can also be directed toward specific antigens in a manner similar to the approaches used to direct immunostimulatory responses. For example, immunosuppressive amounts of superantigen-containing membrane-infiltrating polypeptides can be coated onto cells that contain a specific target antigen. Administering these cells to a mammal can trigger T cell anergy or clonal deletion of the cells having specificity for the target antigen. In this case, the mammal will be unable to recognize the specific antigen and thus will not elicit an immune response against it. This approach is particularly useful for treating autoimmune diseases such as multiple sclerosis and rheumatoid arthritis since autoimmune antigens can be coated onto the same cell containing immunosuppressive membrane-infiltrating polypeptides.

Collectively, the results reported herein establish that superantigens can induce immunostimulation in the absence of MHC II molecules, and that artificially anchoring a superantigen onto a cell surface can substitute for MHC II presentation. Further, low concentrations of superantigen trigger antigen-specific immunostimulatory responses, whereas high concentrations trigger antigen-specific immunosuppression such as T cell anergy.

Superantigen Fragments That Do Not Bind MHC II

The invention provides truncated superantigen fragments that lack the ability to bind MHC II molecules but retain the ability to induce anti-tumor activity. Superantigens having these properties are particularly useful for therapeutic approaches that use superantigens to regulate immune responses. Briefly, superantigens generally direct T cell cytotoxicity against MHC II-positive cells on which they are bound (Kalland T et al., Curr. Top. Micro. Immunol. 174:82-92 (1991)). Thus, any clinically feasible application of superantigen-based anti-tumor strategies will likely require compromising MHC II binding capabilities in order to prevent unwanted cytotoxicity. The truncated superantigen fragments of the invention are novel and useful particularly since the fragments do not exhibit any significant MHC II binding with only a slight decrease in superantigenicity. This is an unexpected result since the manipulation of other superantigens has been shown to either slightly reduce MHC II binding or dramatically reduce superantigenicity, indicating that superantigen structure is sensitive to manipulation. Thus, the invention demonstrates for the first time that superantigens can be separated into distinct domains with each domain retaining its particular activity. Specifically, the experiments described herein demonstrate that a truncated superantigen fragment, soluble TSST (88-194), expressed as a recombinant polypeptide, does not bind MHC II molecules. Further, truncated superantigen fragments as well as membrane-infiltrating polypeptides containing these fragments can induce an anti-tumor response without binding MHC II molecules, thus displaying reduced cytotoxicity against normal MHC II-positive cells.

Biologic activities of the C-terminal domain of TSST1

Residues within the TSST1 C-terminal domain are critical to the superantigenic properties of this molecule (Murray D et al., Infect. Immun. 64:371 (1996); Blanco L et al., Infect. Immun. 58:3020 (1990); and Hurley JM et al., J. Exp. Med. 181:2229 (1995)). H-TSST(88-194) did not bind MHC II molecules, but induced proliferative and cytotoxic responses in vitro and in vivo (see Examples). In the present experiments, the MHC II-binding ability of HK-TSST(88-194) was tested at molar concentrations up to 12-fold greater than that required to elicit a maximal proliferative response, thereby remaining in a biologically relevant concentration range. At concentrations greater than 1 μM, H-TSST(88-194) might also bind MHC II molecules. Nevertheless, [³²P]HK-TSST(88-194) labeled to a high specific activity, did not detectably bind Raji cells at concentrations up to 800 nM (data not shown). These results demonstrate that immunomodulatory actions of H-TSST(88-194) are independent of MHC II binding.

An MHC II-independent response does not necessarily exclude the involvement of antigen presenting cells (APCs) and a myriad of other potential binding molecules. Indeed, APC involvement is probably required. TSST1 was shown to induce T cell proliferation in the complete absence of MHC II molecules as long as APCs were present (Dennig D et al., Cell. Immunol. 171:200 (1996)). Further, about 100-fold greater concentrations of TSST1 were required for an equivalent proliferative response in the absence of MHC II presentation (Dennig D et al., Cell. Immunol. 171:200 (1996)). H-TSST(88-194) induced PBL release of equimolar amounts of TNF-α and TNF-β, produced primarily by monocytes and T cells, respectively. This indicates the probable involvement of both of these cell types upon H-TSST(88-194) stimulation. In addition, cross-linking CD28 molecules with antibodies to provide costimulatory signaling, in the event that H-TSST(88-194) did not efficiently encourage T cell:APC cognate interaction, did not markedly improve the concentration-response curve (data not shown). Although the present invention is not limited to a particular mode of action, these results suggest that H-TSST(88-194)-induced biologic activity is independent of MHC II binding, but involves direct contact between T cells and APCs, and perhaps an alternative, low affinity receptor.

Intact superantigens generally direct T cell-mediated cytotoxicity against MHC II-positive cells on which they are bound (Dohlsten M et al., Immunology 71:96 (1990) and Hermann T et al., J. Immunol. 144:1181(1990)). Immunostimulatory superantigens that are unable to engage MHC II, such as H-TSST(88-194), can be used to develop superantigen-based anti-tumor therapies that avoid directing undesirable toxicity against normal MHC II-positive cells. Administering equimolar amounts of tumor cell-attached H(cys)-TSST1 or H-TSST(88-194) resulted in comparable inhibition of tumor outgrowth. Thus, contrived attachment of a superantigen to the surface of a cell, whether through cross-linking or antibodies, may substitute for MHC II presentation. Collectively, the results indicate that the individual domains of TSST 1 have therapeutic potential. The N-terminal β-barrel, containing residues shown to directly interact with MHC II molecules, can serve as a specific antagonist to ameliorate the acute phase of TSST1-induced TSS. The C-terminal domain, containing residues critical to the superantigenicity of TSST1, can be effectively used in anti-cancer therapies when attached to the surface of tumor cells. These results also demonstrate the separation of function between the two domains of TSST1: the N-terminal domain did not induce proliferation, yet interfered with the stimulatory activities of TSST 1, while the C-terminal domain did not bind MHC II and induced proliferation.

Superantigen Fragments That Bind Only MHC II

The invention also provides truncated superantigen fragments that bind MHC II molecules and inhibit the toxic effects of full-length superantigens. Specifically, these truncated superantigens lack the domains required for superantigenicity. In other words, these fragments are missing the domains that bind the TCR Vβ chain and thus are unable to activate T cells in the same manner as full-length superantigens. Truncated superantigen fragments that bind MHC II molecules but not the TCR Vβ chain can be used to inhibit the toxic effects caused by the full-length superantigen. These truncated superantigens can be made by identifying the individual domains responsible for MHC II binding and TCR Vβ chain binding, removing the domain that binds the TCR Vβ chain, and testing the remaining truncated superantigen for MHC II binding.

Biologic Activities of the N-terminal Domain of TSST1

Since reported body fluid concentrations of TSST1 range from 0.02 to 1.8 nM in patients suffering from TSST1-precipitated TSS (Vergeront JM et al., J. Infect. Dis. 146:456 (1982) and Miwa K et al., J. Clin. Microbiol. 32:539 (1994)), a nonstimulatory version of TSST1 with an intact MHC II binding domain, such as H-TSST(1-87), can specifically antagonize the TSST1 toxin during the acute phase of this disease. There are many reports describing nonstimulatory TSST1 mutants (Murray D et al., Infect. Immun. 64:371 (1996); Blanco L et al., Infect. Immun. 58:3020 (1990); Hurley JM et al., J. Exp. Med. 181:2229 (1995); Murray DL et al., J. Immunol. 152:87 (1994); Kum W et al., J. Infect. Dis. 174:1261 (1996); and Bonventre PF et al., Infect. Immun. 61:793 (1993)). Mutating TSST1 at Asp 132/Gln 136 or His 135 yielded proteins that were nonmitogenic and nonlethal in rabbit infection models for TSS (Murray D et al., Infect. Immun. 64:371 (1996) and Bonventre PF et al., Infect. Immun. 61:793 (1993)). Both of these mutants have intact MHC II binding domains, indicating that TSST1-induced lethality is not a necessary consequence of MHC II binding. Indeed, His 135-mutated TSST1 bound MHC II molecules (Cullen CM et al., Infect. Immun. 63:2141 (1995)) and induced in vitro PBL TNF-α release at concentrations comparable with H-TSST(1-87) (Drynda A et al., Infect. Immun. 63:1095 (1995)). This TNF-α release should not be considered trivial; however, His 135-mutated TSST1 did not precipitate lethal toxic shock in rabbits (Bonventre PF et al., Infect. Immun. 61:793 (1993)) nor in D-galactosamine-sensitized mice (Bonventre PF et al., Infect. Immun. 63:509 (1995)). Thus, any nonstimulatory, MHC II-binding version of TSST1 can be used as a specific antagonist in acute TSST1-precipitated TSS.

The truncated superantigen fragments described herein provide an alternative to the point mutants previously described. In addition, truncated fragments can be associated with less toxicity than the point mutants since the fragments are lacking more than a single amino acid residue that binds the TCR VP chain. In fact, the truncated superantigen fragments can be missing the entire TCR VP chain binding domain. This can be particularly important since the TCR VP chains are variable and polymorphic. For example, some superantigens containing point mutations may remain capable of binding certain TCR VP chain variants.

### EXAMPLES

### Example 1

### Production of Constructs Encoding Membrane-infiltrating Polypeptides

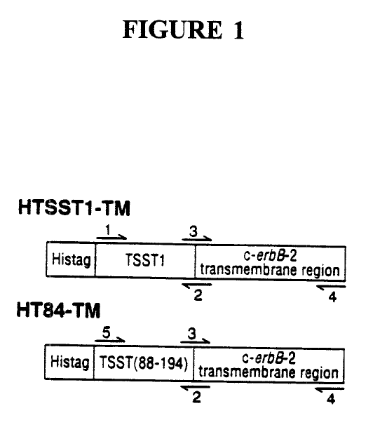

Nucleic acid constructs encoding two different membrane-infiltrating polypeptides were constructed (FIG. 1). The first membrane-infiltrating polypeptide contained a full length superantigen sequence, specifically the 194 amino acid toxic shock syndrome toxin-1 (TSST1). The amino acid sequence of TSST1 is as follows:

STNDNIKDLL DWYSSGSDTF TNSEVLDNSL GSMRIKNTDG SISLIIFPSP YYSPAFTKGE KVDLNTKRTK KSQHTSEGTY IHFQISGVTN TEKLPTPIEL PLKVKVHGKD SPLKYWPKFD KKQLAISTLD FEIRHQLTQI HGLYRSSDKT GGYWKITMND GSTYQSDLSK KFEYNTEKPP INIDEIKTIE AEIN (SEQ ID NO:3)

The second membrane-infiltrating polypeptide contained a truncated version of the TSST1 superantigen sequence (see Example 7 for construction of this version). This truncated version corresponds to the carboxyl-terminus of TSST1 encompassing amino acid residues 88-194 and is designated TSST1(88-194) or T84. The amino acid sequence of T84 is as follows:

VTN TEKLPTPIEL PLKVKVHGKD SPLKYWPKFD KKQLAISTLD FEIRHQLTQI HGLYRSSDKT GGYWKITMND GSTYQSDLSK KFEYNTEKPP INIDEIKTIE AEIN (SEQ ID NO:4)

The coding sequences for TSST1 and T84 were fused to the transmembrane-encoding sequence of proto-oncogene c-erb-B-2 using splice overlap extension PCR (Horton RM et al., Gene 77:61-68 (1989)). This transmembrane-encoding sequence of c-erb-B-2 corresponds to amino acid residues 644-687 and is as follows:

AEQRAS PLTSIISAVV GILLVVVLGV VFGILIKRRQ QKIRKYTM (SEQ ID NO:5)

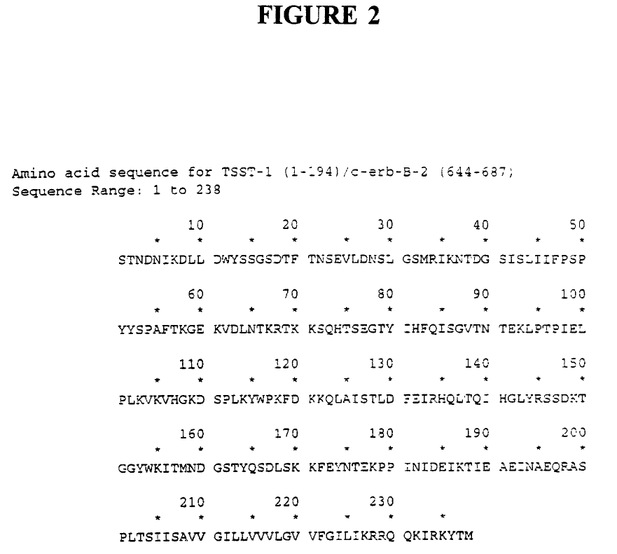

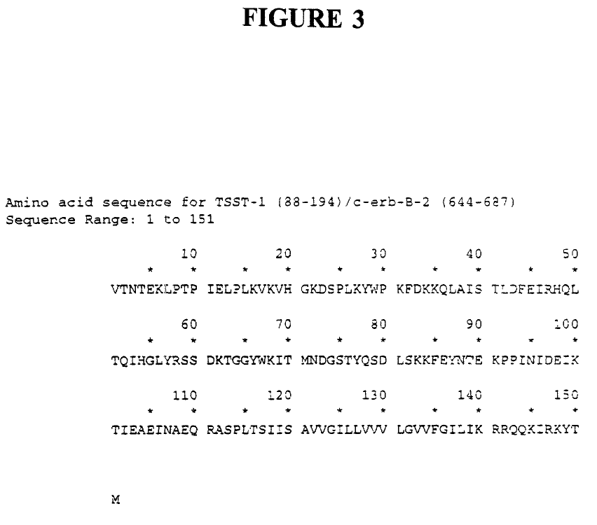

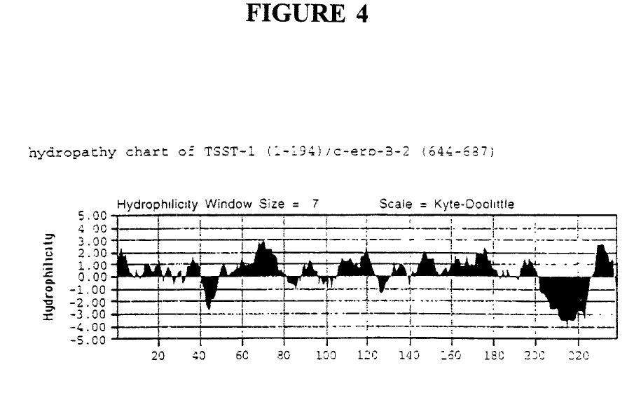

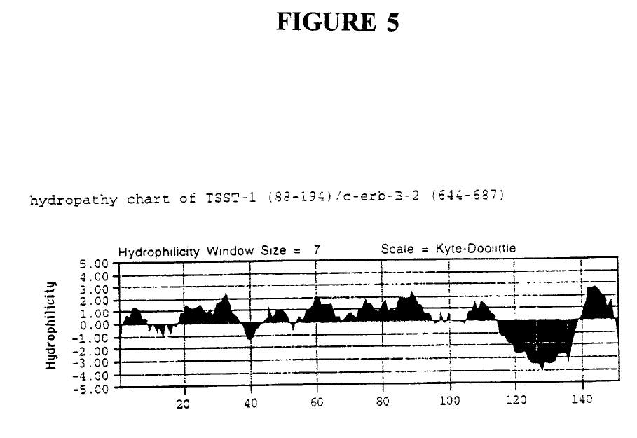

This sequence was attached to the carboxy-terminus of the TSST1 and T84 sequences (FIGS. 2 and 3). Further, this sequence represents one example of a membrane-infiltrating amino acid sequence that endows polypeptides with the ability to insert spontaneously into cell membranes. This particular membrane-infiltrating amino acid sequence is predominantly hydrophobic (FIGS. 4 and 5). The following oligonucleotide primers, with incorporated restriction sites in bold and stop codons underlined, were synthesized by Integrated DNA Technologies (Coralville, Iowa) and used in the splice overlap extension PCR reactions:

(1) 5′-CCCCATATGTCTACAAACGATAATATAAAGGAT-3′ (SEQ ID NO:6)

(2) 5′-TCTCTGCTCGGCACTAGTATTAATTTCTGC-3′ (SEQ ID NO:7)

(3) 5′-GCAGAAATTAATACTAGTGCCGAGCAGAGA-3′ (SEQ ID NO:8)

(4) 5′-CCCCTCGAGTTACATCGTGTACTTCCG-3′ (SEQ ID NO:9)

(5) 5′-CCCCATATGGTTACAAATACTGAA-3′ (SEQ ID NO:10)

The TSST1 and T84 coding sequences were amplified from MNT subclone 6101 (Kreiswirth BN et al., Nature 305:709-712 (1983)) using primers 1 and 2 or primers 5 and 2, respectively. The DNA sequence corresponding to amino acid residues 644-687 of c-erb-B-2, inclusive of the membrane-infiltrating sequence, was amplified from plasmid clone pCER204 (purchased from American Type Culture Collection (ATCC); Rockville, Md.; Yamamoto T et al., Nature 319:230-234 (1986)) using primers 3 and 4. The amplified fragments corresponding to TSST1 (610 base pairs; bp), T84 (320 bp), and c-erb-B-2 (160 bp) were purified and used as templates in subsequent PCR reactions. The chimeric constructs were generated by PCR-amplification from the c-ebb-B-2 fragment together with either the TSST1 or T84 fragments using primers 1 and 4 or primers 5 and 4, respectively. The amplified fragments from the TSST1/c-erb-B-2 reaction (740 bp) and the T84/c-erb-B-2 reaction (450 bp) were individually digested with Nde I and Xho I and ligated into pre-digested prokaryotic expression vector pET 17b Histag (pET 17bH; Mohanraj D et al., J. Immunol. Meth. 182:165-175 (1995)). Positive clones were confirmed through Nde I/Xho I re-digestion and double-stranded DNA sequencing. The enzymes for molecular cloning were purchased from New England Biolabs (Beverly, Mass.). The constructs containing the membrane-infiltrating sequence of c-ebb-B-2 and either TSST1 or T84 are designated TSST1-TM and T84-TM, respectively. In addition, proteins expressed using the pET 17bH expression vector have ten sequential histidine residues at the amino terminus. Thus, TSST1-TM and T84-TM are referred to as HTSST1-TM and HT84-TM, respectively, when using the pET 17bH expression vector.

### Example 2

### HTSST1-TM and HT84-TM Expression

TSST polypeptides are toxic to bacteria that express them, but can be successfully expressed if recombinant polypeptide production is stringently repressed during growth. The expression of HTSST1-TM and HT84-TM in Escherichia coli was adapted from a previously described protocol (Li BY et al., Prot. Expression Purif. 3:386-394 (1992) and Wahlsten JL and Ramakrishnan S, Biotechnol. Appl. Biochem. 24:155-159 (1996)). Briefly, expression vectors containing HTSST-TM and HT84-TM were transformed into E. coli strain BL21 (DE3) which contains the pLysS vector (available from Novagen). This pLysS vector encodes a protein that specifically inhibits the expression of the chromosomally-located T7 polymerase gene. The presence of isopropyl β-D-thiogalactopyranoside (IPTG), however, prevents this protein from being made, thus enabling T7 polymerase transcription.

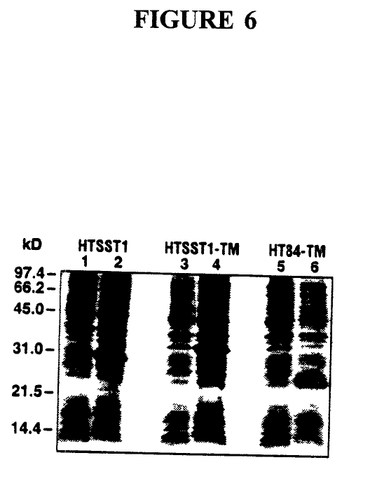

In E. coli host strain BL21 (DE3), the control of T7 polymerase transcription was not completely stringent, resulting in basal expression of polypeptides directed by the T7 promoter (Studier FW and Moffatt BA, J. Mol. Biol. 189:113-130 (1986)). Consequently, considerable levels of HTSST1 were expressed in BL21 (DE3) without induction of T7 polymerase by IPTG (Wahlsten JL and Ramakrishnan S, Biotechnol. Appl. Biochem. 24:155-159 (1996)). Making the growth conditions more stringent, using 2% rather than 0.4% glucose (Li BY et al., Prot. Expression Purif. 3:386-394 (1992)), resulted in the successful expression of TSST1-TM and T84-TM in E. coli host strain BL21 (DE3) pLysS as determined by Coomassie Brilliant Blue-stained 12% SDS-polyacrylamide gel analysis of whole bacterial lysate samples from induced and uninduced cultures (FIG. 6). The lanes depicted in FIG. 6 contain samples from pelleted 1 ml aliquots of 3 ml mini-cultures directly resuspended in 0.5 ml sample buffer (50 mM Tris-HCl, pH 6.8; 100 mM dithioerythritol (DTT); 2% SDS; 0.1% bromophenol blue; 10% glycerol). In each case, 20 μl were loaded per lane. Only IPTG-induced cultures over-expressed recombinant HTSST-TM and HT84-TM polypeptides.

Overnight cultures of HTSST-TM or HT84-TM transformed BL21 (DE3) pLysS cells were inoculated 1:40 into one liter of M9-ZB medium (Studier FW and Moffatt BA, J. Mol. Biol. 189:113-130 (1986)) supplemented with 2% glucose, 100 μg/ml ampicillin, and 34 μg/ml chloramphenicol. Cultures were grown to an absorbance (wavelength 600 nm; A₆₀₀) of 0.8-1.0 with continuous shaking at 37° C. before transcription was induced with IPTG (0.4 mM). Cells were harvested by centrifugation following a 2 hour IPTG induction. The resulting cell pellet was resuspended in 80 ml sonication buffer (300 mM NaCl; 50 mM sodium phosphate, pH 8; 1-2% Triton-X-100) for at least 30 minutes to generate a cell lysate that was frozen at −80° C. The nonionic detergent Triton-X-100 (1-2%) was used to extract HTSST 1-TM and HT84-TM from bacterial cell membranes because refolded TSST polypeptides extracted using denaturing agents such as urea or guanidine-HCl were not functional as determined by a cell proliferation assay.

### Example 3

### HTSST1-TM and HT84-TM Purification

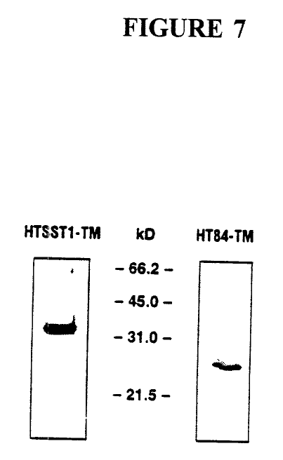

Polypeptides expressed from the pET 17bH expression vector have ten sequential histidine residues at the amino terminus, allowing purification over nickel-agarose. The frozen cell lysate prepared as described in Example 2 was thawed, sonicated for four to six 30 second pulses using a probe sonicator (set to power position 3 on a scale of 1 to 10), and clarified by centrifugation at 28,000× g for 30 minutes. Clearing the lysate of bacterial cell debris enabled the polypeptides to load more efficiently onto the nickel agarose resin when incubated batchwise. This clarified lysate was diluted with an equal volume of sonication buffer and loaded batchwise over 4 hours onto 5 ml bed volume of nickel agarose at 4° C. The nickel agarose resin was rinsed with 50 ml sonication buffer two times, placed in a column support, and rinsed with wash buffer (300 mM NaCl; 50 mM sodium phosphate, pH 8; 1-2% Triton-X-100) until the A₂₈₀ of elute was less than 0.05. This typically required about 100 ml of wash buffer. The sonication and wash solutions can also contain 5-15% glycerol. Nonspecifically bound bacterial host polypeptides were eluted in 25 ml of wash buffer containing 100 mM imidazole. The HTSST1-TM and HT84-TM bound polypeptides were eluted with wash buffer containing 300 mM imidazole in 1 ml fractions, typically between 4 and 20 ml. Positive fractions (A₂₈₀>about 0.1) were pooled and subsequently dialyzed against PBS (137 mM NaCl; 2.7 mM KCl; 4.3 mM Na₂HPO₄; 1.4 mM KH₂PO₄). The purified, dialyzed polypeptides were concentrated using a YM-3 membrane (molecular weight cut-off of about 3,000), aliquoted, and stored at −80° C. Protein concentrations were determined using bicinchoninic acid (BCA) assay reagents (Smith PK et al., Anal. Biochem. 150:76-85 (1985)). Relative protein purity was determined by densitometric measurement of Coomassie Brilliant Blue R-250-stained 12% SDS-polyacrylamide gels. FIG. 7 depicts a representative Coomassie Brilliant Blue-stained 12% SDS-polyacrylamide gel showing purified HTSST1-TM and HT84-TM. This one-step purification strategy yielded 2 mg of HTSST-TM or 0.2 mg of HT84-TM per liter of bacterial culture with an average relative purity of 88%.

### Example 4

### Membrane Insertion of HTSST1-TM and HT84-TM

Superantigens normally bind MHC II molecules (Marrack P and Kappler J, Science 248:705 (1990)). The ability of HTSST1-TM to attach to cells through the spontaneous insertion of the membrane-infiltrating sequence but not by MHC II binding was evaluated on four different MHC II-negative tumor cell lines: EL4, Lewis Lung Carcinoma (LLC), RJ2.2.5, and MA 148. Murine cell lines EL4 and LLC were purchased from ATCC. RJ2.2.5 is a MHC II-negative mutant of the human B cell lymphoma, Raji (Accolla RS, J. Exp. Med. 157:1053-1058 (1983)), that was obtained from Dr. J. M. Boss (Emory University, Atlanta, Ga.) with permission of Dr. R. Accolla (Advanced Biotechnology Center, Genova, Italy). MA 148 is a human ovarian carcinoma cell line. Murine cell lines EL4, LLC, and P815 (mastocytoma cell line; see below) and the human MA 148 cell line remain MHC II-negative even after exposure to IFN-γ (data not shown; Townsend SE et al., Cancer Res. 54:6477-6483 (1994)).

Tumor cells were resuspended to 0.5-1.0×10⁶ cells/ml in medium containing 2.5% fetal bovine serum. Purified HTSST1-TM was added to a final concentration of 0.33-1.67 μM, and incubated for 4 hours at 37° C. Cells were rinsed and analyzed for the presence of HTSST1 on their surfaces by flow cytometry. In the negative control, cells not exposed to HTSST1-TM were incubated with primary and secondary antibodies. Anchored HTSST1-TM was detected with purified IgG from rabbit serum against TSST1 residues 88-194 (αT84), added at a 1:300 dilution with 3% normal goat serum. Bound αT84 was detected with FITC-goat-anti-rabbit IgG (Sigma Chemical Co.; St. Louis, Mo.) added at a 1:200 dilution. Cells were gated for viability using preputium iodide and 2-10×10³ events were measured for positive fluorescence by flow cytometry.

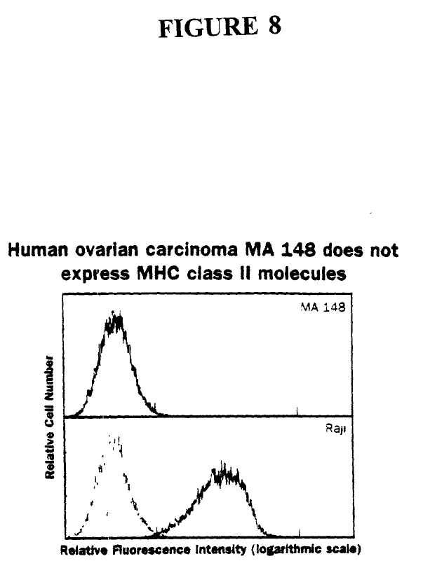

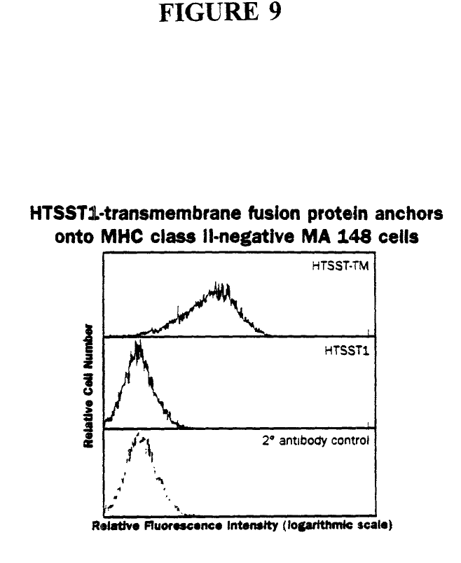

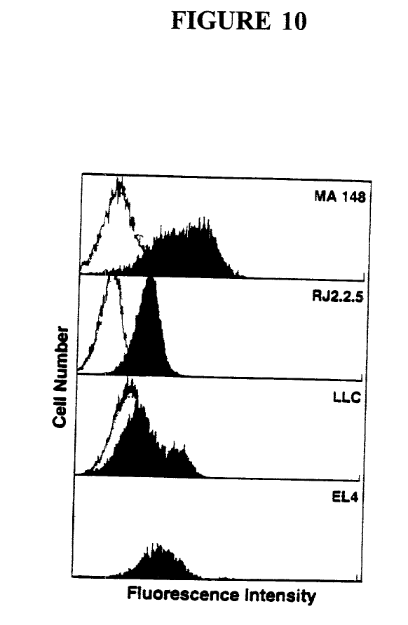

Raji cells express MHC II molecules, whereas MA 148 cells do not (FIG. 8). Flow cytometry results from MA 148 cells treated with HTSST1-TM, HTSST1, and secondary antibody only indicated that HTSST1-TM spontaneously inserted itself into the cell membrane (FIG. 9). The HTSST 1-TM-treated MA 148 cells (750 nM HTSST1-TM polypeptide) were about 70% αT84-positive verses control cells. Further, results from EL4, LLC, RJ2.2.5, and MA 148 cells treated with HTSST1-TM indicated that HTSST1-TM spontaneously inserted itself into the cell membrane of all tumor cell lines tested (FIG. 10). This membrane insertion required the membrane-infiltrating sequence since recombinant native HTSST1 did not associate with RJ2.2.5, MA 148, or LLC cells (FIG. 10).

The tolerance of each cell line to serum deprivation was variable, regardless of exposure to HTSST1-TM. The addition, however, of serum protein concentrations up to 2.5% did not interfere with the membrane inserting ability of HTSST 1-TM and improved cell viability.

Waiting 22 hours after treating MA 148 cells with HTSST 1-TM before preparing the cells for flow cytometry resulted in a level of fluorescence intensity that was 25% of the level observed for MA 148 cells analyzed directly after HTSST1-TM treatment (data not shown). This decrease in HTSST1-TM signal was most likely due to internalization or shedding of HTSST1, because preventing cell division with mitomycin C did not change this result. Interestingly, HTSST1-TM did not significantly insert into cell membranes when incubated at 4° C. (data not shown).

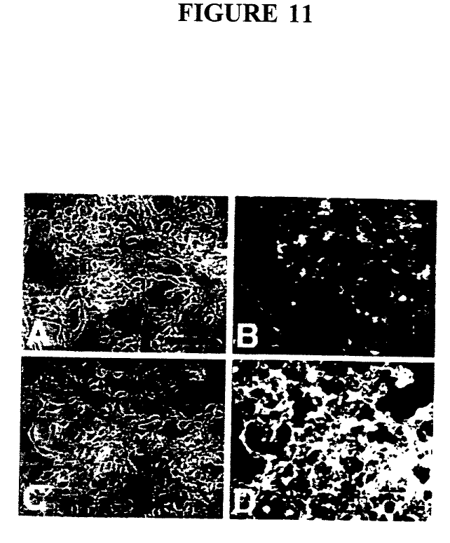

The insertion of HTSST 1-TM into MA 148 cell membranes was also visualized by in situ immunohistochemistry to determine the distribution of HTSST1-TM on the cell surface and the retention of cell integrity after HTSST 1-TM insertion (FIG. 11). MA 148 cells were seeded at low density onto glass coverslips and grown for 24-48 hours. The cells were then incubated for 4 hours at 37° C. in media containing 1 μM HTSST1-TM and 2.5% serum proteins. Cells were rinsed with PBS and fixed for 10 minutes at room temperature (4% paraformaldehyde; 2% sucrose in PBS). The presence of HTSST1-TM was detected using the antibodies described above. Coverslips were mounted onto glass slides. Images were captured with a 10X neofluor objective on a Nikon Diaphot 300 inverted microscope connected to a Photometrics PXL cooled CCD camera (Tucson, Ariz.) using the computer program IPLab Spectrum (Signal Analytics, Vienna, VA.).

MA 148 cells incubated in medium containing 2.5% serum proteins with HTSST 1-TM have HTSST1-TM present on their surface as determined by indirect immunofluorescence, whereas cells not incubated with HTSST1-TM do not (FIG. 11). The phase-contrast exposures demonstrated equal amounts of cells for each condition. In addition, the incubation procedure did not appear harmful to the cells.

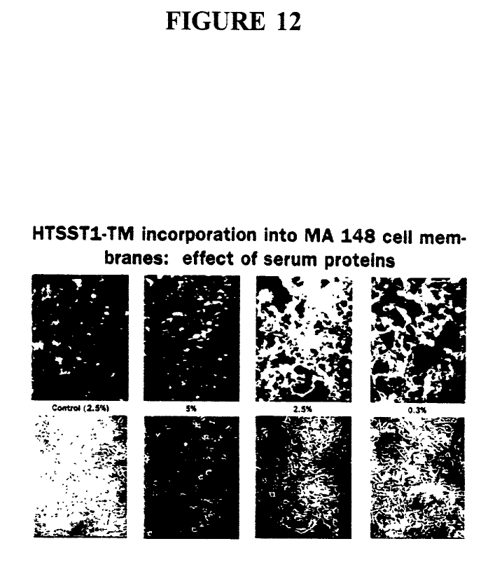

Altering the concentration of serum proteins present during the HTSST1-TM incubation influenced membrane insertion. Incubation of HTSST-TM with cells in the presence of normal media (10% serum) interfered with its insertion into MA 148 cell membranes (data not shown). Decreasing the amount of serum to 2.5% or less, however, allowed for effective and evenly distributed HTSST1-TM insertion into MA 148 membranes (FIG. 12).

### Example 5

### Stimulation of Immune Cells by HTSST1-TM- or HT84-TM-containing Cells

The in vitro biological activity of tumor cells having either HTSST 1-TM or HT84-TM spontaneously inserted into their cell membrane was measured using a lymphocyte proliferation assay. Peripheral blood lymphocytes (PBLs) were isolated from the blood of healthy donors over a Histopaque 1077 gradient (Sigma) and aliquoted to 2×10⁵ cells/well in 96-well U-bottom plates. The PBLs were incubated in 0.2 ml RPMI 1640 medium containing 5% fetal calf serum together with irradiated (10,000 rad), coated or uncoated MA 148 cells at a 1:1 or 1:2 tumor cell to PBL ratio. The MA 148 cells were coated with 1.67 μM of either HTSST1-TM or HT84-TM. MA 148 cells also were coated with five-fold serial dilutions of HTSST1-TM. After incubating the PBL/tumor cell mixture for 72 hours (37° C.; 50% CO₂), cells were labeled with [³H]thymidine (1 μCi/well) for 18 hours prior to harvesting. Incorporation of [³H]thymidine was determined using a liquid-scintillation counter. Results are presented as counts per minute (cpm) for each treatment. Each value represents the average cpm standard deviation of triplicate cultures. Values obtained from lymphocytes alone were subtracted as background and the two-tailed Student's test was used to determine statistical significance.

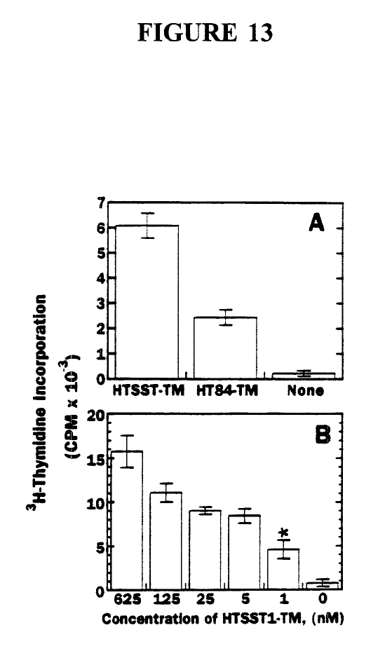

MA 148 cells coated with 1.67 μM of either HTSST1-TM or HT84-TM before being cultured with PBLs (1:1 tumor/lymphocyte ratio) stimulated lymphocyte proliferation (FIG. 13A). Thus, TSST1 polypeptides inserted into a cell membrane are biologically active. Further, the presence of ten amino-terminal histidine residues did not interfere with TSST1 activity.

In an attempt to determine if concentrations below the level of detection by flow cytometry can stimulate immune cells, MA 148 cells were coated with 5-fold serial dilutions of HTSST1-TM before being cultured with PBLs (1:2 tumor/lymphocyte ratio). The minimum concentration of HTSST 1-TM used to coat MA 148 cells that was detected by flow cytometry was 25 nM (data not shown). Tumor cells coated with superantigen concentrations 25-fold less than the minimum level of detection induced a biological response (FIG. 13B). Specifically, preincubating MA 148 cells with as little as 1 nM HTSST1-TM induced significant PBL proliferation versus uncoated MA 148 cells (p<0.004, FIG. 13B), indicating that TSST1 is a powerful immunostimulant when attached to cells. For comparison, directly applying soluble TSST1 at concentrations as low as 2.3 pM induced significant proliferation of human peripheral blood mononuclear cells in vitro (Poindexter NJ and Schlievert PM, J. Infect. Dis. 151:65-72 (1985)). Taken together, the results presented herein indicate that membrane bound HTSST1-TM and HT84-TM can induce lymphocyte proliferation in vitro at very low concentrations.

### Example 6

### Treating a Mammal with HTSST1-TM- or HT84-TM-containing cells

The in vivo biological activity of tumor cells having either HTSST1-TM or HT84-TM spontaneously inserted into their cell membranes was determined using an immunization protocol (Mills C et al., J. Exp. Med. 154:621-630 (1981); Mills C et al., J. Immunol. 149:2709-2714 (1992) and Dye E et al., J. Exp. Med. 154:609-620 (1981)). Murine P815 mastocytoma cells were used since they are well characterized immunogenic tumor cells (North R, Adv. Immunol. 35:89-155 (1984)). P815 tumor cells (1×10⁶ cells/ml) were incubated in serum-free medium alone or with either HTSST 1-TM (0.17 or 1.67 μM) or HT84-TM (0.03, 0.17, or 0.85 μM) as described above. Tumor cells were then rinsed, resuspended at 1×10⁷ cells/ml, and treated with 50 μg/ml Mitomycin C (Mit C) for 1 hour at 37° C. Eight week-old female C57BL/6×DBA/2 (B6D2F1) mice were purchased from Taconic Farms (Germantown, N.Y.). These mice (5 per group) were immunized subcutaneously in the foot with superantigen-coated or control tumor cells (1×10⁷ cells in 50 μl/mouse). At 21 days post immunization, anti-tumor immunity was determined by challenging mice intradermally in the axillary region with 5×10⁵ or 1×10⁶ viable P815 tumor cells. Anti-tumor immunity was determined by measuring the bisecting tumor diameters with calipers every 2-3 days. Moribund mice or mice with ulcerated tumors were sacrificed. All control mice developed tumor. Some mice in the treatment groups did not develop tumors. Of the tumors that did develop, some regressed and others were progressive. Thus, observed differences between control and test groups were evident.

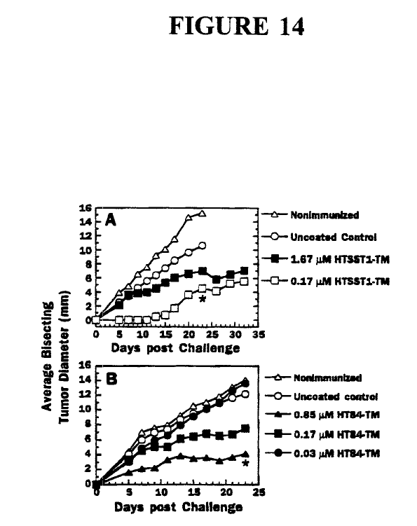

Mice preimmunized with P815 cells incubated with either 1.67 μM or 0.17 μM HTSST1-TM exhibited an anti-tumor immune response (FIG. 14A). Specifically, mice preimmunized with IP815 cells coated with 0.17 μM HTSST1-TM elicited a significant anti-tumor response in comparison to mice preimmunized with uncoated P815 tumor cells (p≦0.03; using the nonparametric Wilcoxon Rank Sum test). Mice for this group had reduced tumor incidence (3 of 5 mice), delayed appearance of tumors that did develop, and the complete regression of one tumor. Mice preimmunized with P815 cells incubated with a higher concentration of HTSST1-TM (1.67 μM) all initially developed tumor. One tumor, however, completely regressed and two others partially regressed, as indicated in the smaller average tumor diameter compared to treatment with uncoated, control cells (FIG. 14A)>The observed anti-tumor immunity was most likely tumor specific since the anti-tumor response was dose-dependant with the higher dose of HTSST1-TM being less effective. The fact that the tumor challenge site was distant from the site of immunization is also suggestive of tumor specificity. Further, mice with tumors that completely regressed (one from each test group) were re-challenged with 1×10⁶ parental P815 cells 87 days postimmunization and neither developed tumor (data not shown).

Mice preimmunized with P815 cells coated with HT84-TM also exhibited an anti-tumor immune response. Specifically, mice preimmunized with P815 cells incubated with 0.85 μM HT84-TM elicited significant anti-tumor immunity in comparison to mice preimmunized with uncoated P815 tumor cells (p≦0.02). Of the five mice in this group, one did not develop tumor and two developed tumors that completely regressed. All mice preimmunized with P815 cells incubated with 0.17 μM HT84-TM initially developed tumor, but two tumors completely regressed and one partially regressed (FIG. 14B). When comparing the anti-tumor responses elicited by equimolar concentrations of HT84-TM and HTSST 1-TM (0.17 μM of HTSST1-TM or HT84-TM), HTSST1-TM-coated cells were more potent immunogens, correlating with the in vitro proliferation experiments (FIG. 13). Taken together, these results indicate that HTSST1-TM- or HT84-TM-coated P815 tumor cells stimulate systemic, memory anti-tumor immunity. In addition, lower concentrations of TSST1 are more effective than higher concentrations.

Coating tumor cells with smaller amounts of HTSST1-TM is not only more desirable for minimizing toxicity, but also resulted in better systemic anti-tumor immunity. Specifically, P815 tumor cells coated with 0.17 μM HTSST1-TM significantly inhibited growth and onset of a parental tumor challenge whereas P815 cells incubated with 1.67 μM HTSST1-TM was less effective.

Collectively, the results reported herein establish that superantigens can induce immunostimulation in the absence of MHC II molecules, and that artificially anchoring a superantigen onto a cell surface can substitute for MHC II presentation. Further, low concentrations of superantigen trigger antigen-specific immunostimulatory responses, whereas high concentrations trigger antigen-specific immunosuppression such as T cell anergy.

### Example 7

### Construction and Characterization of TSST1 Fragments

Structurally, TSST1 is composed of two distinct domains (Acharya KR et al., Nature 367:94 (1994) and Prasad GS et al., Biochemistry 32:13761 (1993)). In an attempt to determine if the proposed functional domains of the TSST1 molecule are separable, the TSST1 N-terminal residues 1-87 (TSST(1-87)) and C-terminal residues 88-194 (TSST(88-194)) were expressed and analyzed individually. TSST(1-87) has the following amino acid sequence:

STNDNIKDLL DWYSSGSDTF TNSEVLDNSL GSMRIKNTDG SISLIIFPSP YYSPAFTKGE KVDLNTKRTK KSQHTSEGTY IHFQISG (SEQ ID NO:11)

1. Construction, Expression, and Purification of TSST Fragments

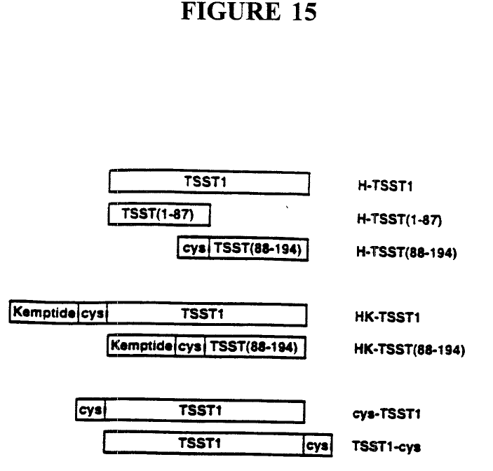

The native TSST1, N-terminal TSST(1-87), and the C-terminal TSST(88-194) constructs are depicted schematically in FIG. 15. The prokaryotic expression vector. pET 17bH, was used to express each construct resulting in the addition of an amino terminal histidine (H-) tag. The histidine tag facilitates polypeptide purification as described in Example 3. In addition, TSST1 and TSST(88-194) constructs were made to contain a Kemptide (K-) sequence, a specific PKA phosphorylation sequence that serves as a ³²p radiolabeling site (Kemp BE et al., Proc. Natl. Acad. Sci. USA 73:1038 (1976) and Mohanraj D et al., Protein Expr. Purif. 8:175 (1996)). These polypeptides are referred to as HK-TSST1 and HK-TSST(88-194).

Cysteine residues aid in the chemical cross-linking of polypeptides to the surface of cells through the formation of disulfide linkages. Since native TSST1 has no cysteine residues, a single cysteine residue was inserted into either the N- or C- terminus (cys-TSST1 or TSST1-cys) of TSST1. In addition, N-terminal cysteines were inserted into H-TSST(88-194), HK-TSST1, and HK-TSST(88-194).

All sequences were PCR amplified from MNT subclone 6101 (Kreiswirth B et al., Nature 305:709 (1983)) using primers (Integrated DNA Technologies; Coralville, Iowa) designed to incorporate 3′ Nde I and 5′ Hind III restriction sites. The primers used to amplify the mature coding region of TSST1 are described elsewhere (Wahlsten JL and Ramakrishnan S, Biotechnol. App. Biochem. 24:155 (1996)). The downstream primer for TSST(1-87) was 5′-CGGAAGCTTTTAGCCACTTATTTGGAA-3′ (SEQ ID NO:12). The upstream primer for TSST(88-194), also including a 5′ end cysteine codon, was 5′-CCCATATGTGCGTTACAAATACTGAAAAATAACCT-3′ (SEQ ID NO:13). The upstream primers used to amplify K-TSST1 and K-TSST(88-194), each having the Kemptide sequence and a cysteine codon, were as follows: K-TSST1, 5′-CCCCATATGTGCCTGCGTCGTGCGAGCCTGGGTTCTACAAACGATAAT-3′ (SEQ ID NO: 14); K-TSST(88-194), 5′-CCCCATATGTGCCTGCGTCGTGCGAGCCTGGGTGTTACAAATACTGAA -3∝ (SEQ ID NO:15). The upstream cys-TSST1 and downstream TSST 1-cys primers were 5′-CCCCATATGTGCTCTACAAACGATAATATAAAG-3′ (SEQ ID NO:16) and 5′-CTCGGTACCAAGCTTTTAGCAATTAATTTC-3′ (SEQ ID NO: 17), respectively.

All amplified sequences were subcloned into pET 17bH, sequenced, and expressed in E. coli host strain BL21 (DE3), according to a previously described protocol (Wahlsten JL and Ramakrishnan S, Biotechnol. App. Biochem. 24:155 (1996) and Li BY et al., Protein Expr. Purif. 3:386 (1992)). Homogeneous monomeric polypeptide preparations of unlinked cysteine-bearing constructs were made by treatment with DTT and then iodoacetamide. Relative polypeptide purity of all H-TSST1 constructs was assessed by densitometric measurements of Coomassie Brilliant Blue-stained 12% SDS-polyacrylamide gels. In each case, a single band of at least 95% purity was observed.

2. Binding of TSST1 Fragments to MHC Class II Molecules

To compare binding of H-TSST1 and H-TSST(88-194) to MHC II molecules, these polypeptides were expressed with a N-terminal Kemptide sequence. Kemptide is a specific phosphorylation sequence for cAMP-dependent protein kinase A (PKA; Kemp BE et al., Proc. Natl. Acad. Sci. USA 73:1038 (1976) and Pearson RB and Kemp BE, Methods Enzymol. 200:62 (1991)), and therefore serves as a specific ³²P radiolabeling site. The purification, labeling, and biologic activity of HK-TSST1 have been described (Mohanraj D et al., Protein Expr. Purif. 8:175 (1996)). For labeling, 83 pmol HK-TSST1 (2 μg) or HK-TSST(88-194) (1.3 μg) was incubated with equimolar amounts of [γ-³²P] ATP (3000 Ci/mmol) and 20-30 Units PKA for 30 minutes at 30° C. in 60 μl buffer (50 mM MOPS, pH 7; 10 mM MgCl₂). Labeled polypeptide was separated from free nucleotide using a spin column and the amount of radioactivity for the trichloroacetic acid (TCA)-precipitable material was measured. The specific activity of labeled HK-TSST1 and HK-TSST(88-194) was 1100 and 2100 μCi/nmol, respectively.

Serial two-fold dilutions of labeled [³²P]HK-TSST1 or [³²P]HK-TSST(88-194) were incubated with 3×10⁵ MHC II-positive or MHC II-negative cells for 1 hour on ice in 50 μRPMI 1640 containing 5% bovine calf serum and 0.02% NaN₃. Specific binding was blocked by addition of a 100-fold molar excess of the same unlabeled polypeptide. Cells were rinsed three times and radioactivity of aliquots containing 8.5×10⁴ cells measured in a scintillation counter.

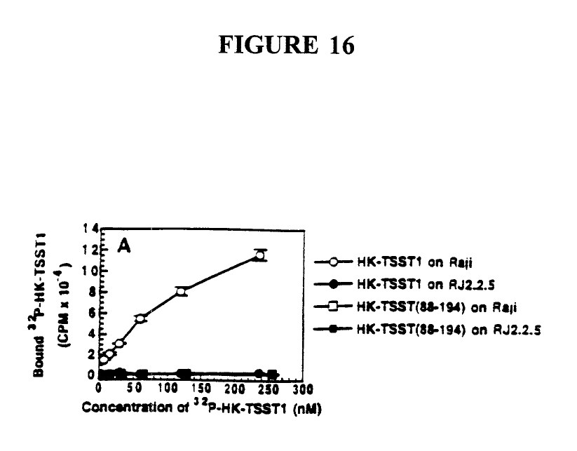

Labeled [³²P]HT-TSST1 clearly bound MHC II-positive Raji cells, but did not bind to MHC II-deficient RJ2.2.5 cells. In contrast, labeled [³²P]HT-TSST(88-194) did not bind to either Raji or RJ2.2.5 cells, even when present at 250 nM (FIG. 16). In a separate experiment, H-TSST(88-194) did not detectably bind Raji cells when present at 800 nM (data not shown). These results demonstrate that H-TSST(88-194) does not bind MHC II molecules.

3. Stimulation of Immune Cells in vitro by TSST1 Fragments

The biologic activity of H-TSST1, H-TSST(88-194), and H-TSST(1-87) was measured in proliferation assays using human PBLs. Gradient-isolated PBLs were plated at 2×10⁵ in 0.2 ml RPMI 1640 containing 10% FBS, 15 μg/ml polymyxin B sulfate, and serial dilutions of polypeptide. Cultures were incubated for 72 hours, labeled with [³H]thymidine (83Ci/mmol), and harvested 18 hours later. The incorporated radioactivity was measured in a scintillation counter.

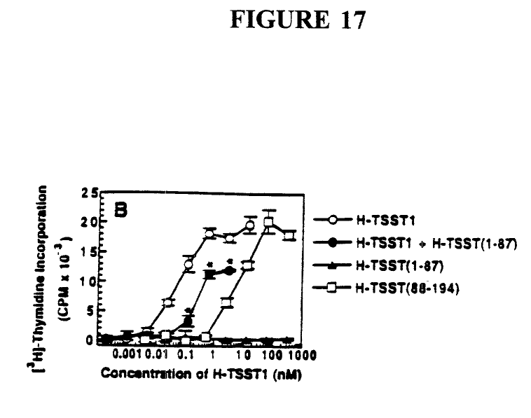

The addition of up to 320 nM H-TSST(1-87) did not induce proliferation of PBLs (FIG. 17). Since TSST(1-87) did not induce proliferation and contains residues that directly interact with MHC II molecules, the ability of TSST(1-87) to inhibit TSST1-induced proliferation was tested. Indeed, a 100-fold molar excess of H-TSST(1-87) incubated with H-TSST1 shifted the H-TSST1 concentration-response curve down by 10-fold, and reduced the maximum H-TSST1-induced proliferation by 30% (p<0.001, FIG. 17). These data suggest that H-TSST(1-87) does not induce proliferation and interferes with proliferation induced by intact H-TSST1. It is possible, however, that H-TSST(11-87) may induce proliferation if cultured at greater concentrations.

Although H-TSST(88-194) does not engage MHC II molecules, it induces proliferation of PBLs in vitro. The maximum proliferation of PBLs stimulated by H-TSST (88-194) was equal to that induced by H-TSST1, although 100-fold greater molar concentrations were required (FIG. 17). H-TSST(88-194) induced maximum PBL proliferation at 65 nM, a concentration 12-fold less than the maximum concentration tested (800 nM) to demonstrate its inability to bind MHC II molecules (FIG. 17 and data not shown). These results demonstrate that H-TSST(88-194) stimulates PBL proliferation independent of MHC II binding.

4. Stimulation of Cytotoxic Immune Cells in vitro by TSST1 Fragments

The ability of H-TSST1 and H-TSST(88-194) to induce cytotoxic activity of PBLs against the human ovarian carcinoma cell line, MA 148, was evaluated in a 4 hour ⁵¹Cr release assay. PBLs (1×10⁶/ml) were prestimulated with H-TSST1 (0.1 μg/ml) or H-TSST(88-194) (1 μg/ml) for 4 days. On day 4, MA 148 cells were labeled with ⁵¹Cr (300 μCi/5×10⁵ cells) for 3 hours, rinsed, and plated at 4000 cells/well in 96-well U-bottom plates. PBLs were thoroughly rinsed of free polypeptide, serially diluted (1/3), and added at different effector:target cell ratios (E:T ratios). Effector cells were not added to background lysis control wells, whereas 0.2% Triton X-100 was added to the 100% lysis control wells. After 4 hours at 37° C., radioactivity released into supernatants was measured in a gamma counter.

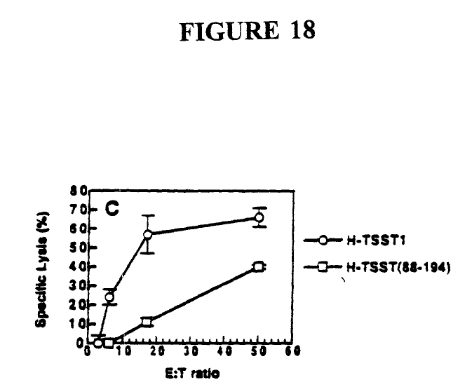

Human PBLs prestimulated with either H-TSST1 or H-TSST(88-194) were cytotoxic against MA 148 cells; however, about a five-fold greater E:T ratio was required for the H-TSST(88-194)-stimulated PBLs to elicit a comparable response (FIG. 18) compared to H-TSST1.

5. TCR Vβ-Specific Proliferation by TSST1 Fragments

Superantigens induce selective proliferation of T cells bearing certain TCR Vβ elements (Marrack P and Kappler J, Science 248:705 (1990)). Primarily, human T cells that express TCR Vβ2 proliferate upon exposure to TSST1 (Choi Y et al., J. Exp. Med. 172:981 (1990)). Flow cytometry and monoclonal antibodies (mAbs) specific for Vβ2 were used to determine the TCR Vβ expansion profiles of H-TSST1- and H-TSST(88-194)-stimulated PBLs. PBLs were incubated (1×10⁶ cells/ml) with H-TSST1 (5 ng/ml) or H-TSST(88-194) (500 ng/ml) for 72 hours, washed, and then resuspended in an equal volume of fresh RPMI 1640 containing 50 Units/ml human rIL-2 (gift from Cetus-Perkin-Elmer; Emeryville, Calif.). After 24 hours, cells were prepared for flow cytometry. TCR Vβ2, Vβ5, or Vβ8 elements were detected with appropriate primary antibodies (2 μg/test) diluted in medium containing 3% normal goat serum. Anti-human V⊕2 mAb clone TCRBV2S1 was purchased from Immunotech/Coulter (Miami, Fla.). Anti-human Vβ5 (clone MH3-2) and anti-human Vβ8 (clone JR2) were purchased from PharMingen (San Diego, Calif.). The mAbs specific for Vβ5 and Vβ8 were used as negative controls. Bound primary Antibodies were detected with FITC-labeled goat anti-mouse polyvalent Ig (Sigma; used at 1:250 dilution). Cells were then incubated with phycoerythrin-labeled mouse anti-human CD3 (Sigma; clone UCHT-1, used at 1:20 dilution). Data were presented as the percentage of CD3-positive lymphocytes or blasts, as indicated by forward and side scatter, that also stain positive for TCR Vβ2, Vβ5, or Vβ8. Values of positively staining cells in the presence of only secondary antibody were subtracted as background before making the calculations.

PBLs gated for expression of CD3 and Vβ2, Vβ5, or Vβ8 elements stained equivalently, whether stimulated with H-TSST1 or H-TSST(88-194). The percentage of CD3⁺ blasts expressing Vβ2 tripled verses nonstimulated controls, and a correlating decrease of TCR Vβ2⁺ cells remained in the resting CD3⁺ lymphocyte population. This result implies that the majority of T cells bearing Vβ2 were stimulated to become blasts upon exposure to H-TSST1 or H-TSST(88-194) (Kappler JB et al., Science 244:811 (1989)). By comparison, the percentage of CD3+blasts expressing Vβ5 doubled, and the proportions of Vβ8-expressing CD3+cells did not appreciably change when stimulated with either H-TSST1 or H-TSST(88-194) (Table I). Nominal expansion of Vβ5-expressing T cells upon exposure to TSST1 has been previously observed (Kappler JB et al., Science 244:811 (1989) and Choi Y et al., Proc. Natl. Acad. Sci. USA 86:8941 (1989)). These results demonstrate that H-TSST1 and HTST(88-194) stimulated T cells similarly.

TABLE I
T cell receptor Vβ-specific proliferation stimulated by H-TSST1 and H-
TSST(88-194)^{a}

| | CD3⁺ Lymphocytes Binding | | | CD3⁺ Blasts Binding | | |
|---|---|---|---|---|---|---|
| | Anti-Vβ (%) | | | Anti-Vβ (%) | | |
| Stimulus | Vβ2 | Vβ5 | Vβ8 | Vβ2 | Vβ5 | Vβ8 |
| None | 9.6 | 2.3 | 4.1 | | | |
| H-TSST1 | 5.1(0.5)^{b} | 2.0(0.9) | 4.1(1.0) | 33.0(3.4) | 4.5(2.0) | 4.6(1.1) |
| H-TSST | 5.8(0.6) | 3.2(1.4) | 4.2(1.0) | 29.0(3.0) | 5.4(2.4) | 5.2(1.3) |
| (88-194) | | | | | | |

^{a}PBL were incubated with 5 ng/ml H-TSST1 or 500 ng/ml H-TSST(88-194) for 72 hours, rinsed, and then incubated in medium containing 50 Units/ml rIL-2 for 24 hours. Percentages of cells staining positive with addition of only secondary antibody were subtracted as background.
^{b}Fold differences versus unstimulated controls are in parentheses, calculated as the percentage of lymphocytes or blasts that bound an anti-Vβ antibody after stimulation divided by the percentage of lymphocytes that bound the anti-Vβ before stimulation.

6. TNF-α and TNF-β Release by Immune Cells Stimulated with TSST1 Fragments

TSST-1 potently induces release of TNF-α and TNF-β (Jupin CS et al., J. Exp. Med. 167:752 (1988) and Hackett SP and Stevens DL, J. Infect. Dis. 168:232 (1993)). This TNF release can cause lethal TSS (Miethke T et al., Eur. J. Immunol. 23:1494 (1993)). The ability of H-TSST1, H-TSST(88-194), and H-TSST(1-87) to stimulate the release of TNF-α and TNF-β by PBLs was measured using immunoassay kits (R&D Systems; Minneapolis, Minn.). PBLs were incubated (1×10⁶ cells/ml) with H-TSST1 (5 ng/ml), H-TSST(88-194) (500 ng/ml), H-TSST(1-87) (240 ng/ml), or H-TSST1 plus H-TSST(1-87) in RPMI 1640 containing 10% FBS and 15 μg/ml polymyxin B sulfate for about 72 hours. Supernatants were clarified of cellular debris and diluted threefold before quantifying TNF release by enzyme-linked immunosorbent assay (ELISA) following the protocols recommended by the manufacturer.

Since TSST(1-87) did not induce proliferation and inhibited TSST 1-induced proliferation, the ability of TSST(1-87) to induce TNF release as well as inhibit TSST1-induced TNF release was determined. Unexpectedly, PBL cultures incubated with as little as 0.5 nM H-TSST(1-87) elicited nearly 300 pg/ml TNF-α release. The highest concentration of H-TSST(1-87) tested (40 nM), however, was unable to induce detectable amounts of TNF-P release (Table II). Further, the concentrations of H-TSST1-induced TNF-a and TNF-P released through H-TSST1 induction were reduced by 40 and 85%, respectively, upon incubation with a 200-fold molar excess of H-TSST(1-87) with H-TSST1 (p<0.001 for inhibition of both TNF isoforms, Table II).

Human PBLs incubated with an H-TSST(88-194) concentration that induces proliferation (32 nM) released large amounts of both TNF-A and TNF-P (Table II), comparable with H-TSST1-induced release. At 100-fold lower concentrations (0.5 and 0.1 nM), in which only H-TSST1 is maximally active, H-TSST(88-194) did not elicit detectable amounts of TNF-α or TNF-β production. Taken together, these results demonstrate that H-TSST(88-194) stimulates release of both TNF isoforms, concomitant with proliferation. Although H-TSST(1-87) stimulates release of TNF-α, a molar excess of H-TSST(1-87) significantly inhibits H-TSST1-stimulated release of both TNF isoforms.

TABLE II
TNF-α and TNF-β released upon stimulation with H-TSST1,
H-TSST(88-194), or H-TSST(1-87)^{a}

Concentration of TNF(pg/ml)

| Stimulus | TNF-α | TNF-β |
|---|---|---|
| None | <4.4^{b} | <16 |
| H-TSST1 (0.2 nM) | 1271 (±22)^{c} | 1269 (±14) |
| H-TSST1 (0.2 nM) + | 741 (±5)^{d} | 192 (±14)^{d} |
| H-TSST1(1-87) (40 nM) | | |
| H-TSST(1-87) (40 nM) | 437 (±14) | <16 |
| H-TSST(1-87) (0.5 nM) | 295 (±12) | <16 |
| H-TSST(1-87) (0.1 nM) | <4.4 | <16 |
| H-TSST(88-194) (32 nM) | 1694 (±27) | 1704(±44) |
| H-TSST(88-194) (0.5 nM) | <4.4 | <16 |
| N-TSST(88-194) (0.1 nM) | <4.4 | <16 |

^{a}PBLs were incubated with indicated concentrations of different H-TSST1 constructs for 84 hours. The supernatants were clarified of cellular debris, and released TNF-α or TNF-β was measured by ELISA.
^{b}The minimum detection limit was 4.4 pg/ml for TNF-α and 16 pg/ml for TNF-β.
^{c}SD of triplicate measurements are in parentheses.
^{d}p < 0.001, two-tailed Student's test.

7. Stimulation of Anti-Tumor Cytotoxicity in vivo

In an attempt to determine the ability of cell-anchored TSST1 to induce an anti-tumor response in vivo, TSST1 polypeptides were chemically cross-linked to tumor cells that were in turn injected into mice. LLC cells (6.25×10⁶ cells/ml) were incubated in Hank's balanced salt solution (HBSS) containing 1 mM heterobifunctional cross-linker, Sulfo-LC-SMPT (Pierce; Rockford, Ill.), for 1 hour at 37° C. Cells were thoroughly rinsed of unbound cross-linker and then incubated for 5 hours at 37° C. at the same cell concentration with 16 μM cys-TSST1, TSST1-cys, or H-TSST(88-194) in serum-free, methionine-free, and cysteine-free high glucose Delbecco's Modified Eagle's Medium (DMEM). Negative control cells were incubated in serum-free high glucose DMEM to allow disulfide exchange with cysteine. Cells were rinsed of unbound polypeptide four times with 15 ml DMEM, irradiated (5000 rad), and directly prepared for injection into mice. Approximately 32 μg H(cys)-TSST1 or 20 μg H-TSST(88-194) (1.3 nmol) was linked to 5×10⁵ cells.

The ability of cell surface-attached cys-TSST1, TSST1-cys, or H-TSST(88-194) to induce a local anti-tumor response was measured in an in vivo tumorigenicity assay. Eight- to ten-week old female C57BL/6 mice (Charles River Laboratories; Wilmington, Mass.) were coinoculated subcutaneously into the left side of the shaved back with 5×10⁵ parental LLC cells and 5×10⁵ coated, irradiated LLC cells. Each treatment group contained 6 mice. Bisecting tumor diameters were measured with calipers every 2 to 4 days. Moribund mice or mice with ulcerated tumors were killed. Because of morbidity, the experiment ended on day 26 postinoculation. Surviving mice were still measured through day 32, and mice without tumor burden were monitored through day 48. The nonparametric Wilcoxon Rank Sum test was used to assess significant differences between treated and control groups.

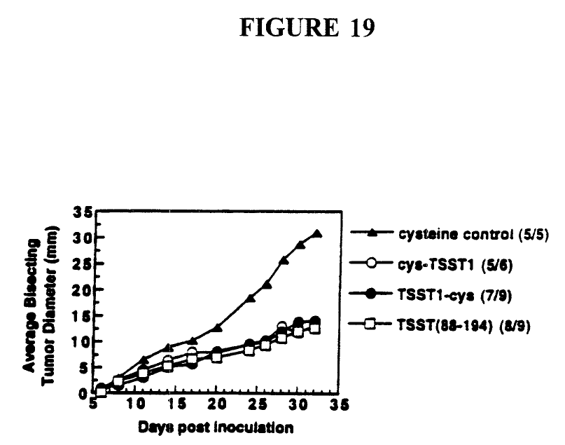

Tumor cells coated with cys-TSST1, TSST1-cys, or H-TSST(88-194) significantly inhibited the outgrowth of coinjected parental tumor cells verses control-treated mice (p<0.02 on day 26, FIG. 19). All control mice developed tumor. Some mice treated with superantigen-coated tumor cells did not develop tumors and the tumors that did develop were progressive. Thus, the average diameter values reflect a reduced incidence and hindered outgrowth of tumor in the treatment groups. The anti-tumor response was independent of N- (cys-TSST1) or C-terminal (TSST1-cys) TSST1 attachment or the presence of MHC II binding domain (FIG. 19).

In summary, the characterization of the TSST1 N- and C-terminal domains revealed that (a) the N-terminal domain did not induce proliferation or TNF-β release, but low concentrations elicited TNF-α release; (b) molar excesses of the N-terminal domain inhibited proliferation and TNF release induced by intact TSST1; and (c) the C-terminal domain did not bind MHC II, but stimulated Vβ-selective proliferation, anti-tumor cytotoxicity, and release of both TNF isoforms.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid molecule encoding a polypeptide, said polypeptide comprising a membrane-infiltrating amino acid sequence and a selected amino acid sequence heterologous to said membrane-infiltrating amino acid sequence.

2. The nucleic acid molecule of claim 1, wherein said polypeptide further comprises an amino acid sequence that facilitates purification of said polypeptide.

3. The nucleic acid molecule of claim 1, wherein said membrane-infiltrating amino acid sequence comprises a transmembrane domain.

4. The nucleic acid molecule of claim 3, wherein said transmembrane domain comprises the transmembrane domain of c-erb-B-2.

5. The nucleic acid molecule of claim 4, wherein said c-erb-B-2 transmembrane domain comprises the amino acid sequence of SEQ ID NO:5.

6. The nucleic acid molecule of claim 1, wherein said selected amino acid sequence comprises a superantigen.

7. The nucleic acid molecule of claim 6, wherein said superantigen comprises a bacterial toxin.

8. The nucleic acid molecule of claim 7, wherein said bacterial toxin comprises toxic shock syndrome toxin.

9. The nucleic acid molecule of claim 8, wherein said toxic shock syndrome toxin comprises an amino acid sequence selected from the group consisting of SEQ ID NO:3 and SEQ ID NO:4.

10. The nucleic acid molecule of claim 6, wherein said superantigen comprises a superantigen having reduced MHC II binding.

11. The nucleic acid molecule of claim 10, wherein said superantigen comprises a superantigen that lacks MHC II binding.

12. The nucleic acid molecule of claim 11, wherein said superantigen comprises the amino acid sequence of SEQ ID NO:4.

13. A host cell comprising a nucleic acid molecule encoding a polypeptide, said polypeptide comprising a membrane-infiltrating amino acid sequence and a selected amino acid sequence heterologous to said membrane-infiltrating amino acid sequence, and wherein said polypeptide is expressed by said host cell.

14. The host cell of claim 13, wherein said host cell comprises a prokaryotic cell.

15. A polypeptide comprising a membrane-infiltrating amino acid sequence and a selected amino acid sequence heterologous to said membrane-infiltrating amino acid sequence.

16. A cell comprising a polypeptide, said polypeptide comprising a membrane-infiltrating amino acid sequence and a selected amino acid sequence heterologous to said membrane-infiltrating amino acid sequence.

17. The cell of claim 16, wherein said cell is free of nucleic acid encoding said polypeptide.

18. The cell of claim 16, wherein said cell comprises a eukaryotic cell.

19. The cell of claim 18, wherein said eukaryotic cell comprises a mammalian cell.

20. The cell of claim 16, wherein said cell is a tumor cell.

21. A method of altering the surface of a cell, said method comprising contacting said cell with a polypeptide, wherein said polypeptide comprises a membrane-infiltrating amino acid sequence and a selected amino acid sequence.

22. The method of claim 21, wherein said selected amino acid sequence is heterologous to said membrane-infiltrating amino acid sequence.

23. A method of treating a mammal, said method comprising administering cells to said mammal, said cells comprising a polypeptide, said polypeptide comprising a membrane-infiltrating amino acid sequence and a selected amino acid sequence heterologous to said membrane-infiltrating amino acid sequence.

24. The method of claim 23, wherein said cells are free of nucleic acid encoding said polypeptide.

25. The method of claim 23, wherein said cells comprise tumor cells.

26. The method of claim 25, wherein said tumor cells are from said mammal.

27. The method of claim 23, wherein said polypeptide comprises a superantigen.

28. A nucleic acid molecule encoding a truncated superantigen lacking the ability to bind MHC class II molecules while retaining the ability to induce an anti-tumor activity.

29. The nucleic acid molecule of claim 28, wherein said truncated superantigen comprises the amino acid sequence of SEQ ID NO:4.

30. A nucleic acid molecule encoding a truncated superantigen that binds MHC class II molecules and inhibits the toxic effects of a non-truncated superantigen.

31. The nucleic acid molecule of claim 30, wherein said truncated superantigen comprises the amino acid sequence of SEQ ID NO: 111.

32. A method of treating a mammal, wherein said method comprises administering to said mammal a truncated superantigen that binds MHC class II molecules and inhibits the toxic effects of a non-truncated superantigen.
